(19) United States
(12) Patent Application Publication
(10) Pub. No.: US 2023/0330069 A1
(43) Pub. Date: Oct. 19, 2023

(54) USE OF THIAZOLIDES AGAINST CORONAVIRUSES

(71) Applicant: Romark Laboratories, L.C., Tampa, FL (US)

(72) Inventor: Jean-Francois Rossignol, St. Petersburg, FL (US)

(73) Assignee: Romark Laboratories, L.C., Tampa, FL (US)

(21) Appl. No.: 18/022,963

(22) PCT Filed: Aug. 23, 2021
(86) PCT No.: PCT/US2021/047128

§ 371 Date: Feb. 23, 2023

Related U.S. Application Data
(60) Provisional application No. 63/069,313, filed on Aug. 24, 2020.

(51) Int. Cl. A61K 31/426; A61P 31/14

(52) U.S. Cl. CPC A61K 31/426; A61P 31/14

(57) ABSTRACT

Disclosed is use of thiazolides, such as nitazoxanide and/or tizoxanide, against viruses belonging to the Coronaviridae family, such as viruses belonging to the Orthocoronavirinae subfamily.

### RELATED APPLICATIONS

The present application claims priority to U.S. provisional patent application 63/069,313 filed Aug. 24, 2020 titled “Use of Thiazolides against Coronaviruses,” which is incorporated by reference herein for all purposes.

### FIELD

The present disclosure relates to thiazolides and more specifically to use of thiazolides, such as nitazoxanide and/or tizoxanide, against viruses belonging to the Coronaviridae family, such as viruses belonging to the Orthocoronavirinae subfamily.

### SUMMARY

One embodiment is a method of treating an illness caused by a virus belonging to the Coronaviridae family, comprising administering to a subject in need thereof an effective amount of a thiazolide agent to produce an effective concentration of tizoxanide in a plasma of the subject, wherein the subject displays one or more symptoms of the illness.

Another embodiment is a method of preventing a viral respiratory illness, comprising administering to a subject, who does not display a symptom of the viral respiratory illness, an effective amount of a thiazolide agent to produce an effective concentration of tizoxanide in a plasma of the subject.

Yet another embodiment is a method of treating a mild or moderate illness caused by severe acute respiratory syndrome coronavirus 2 (SARS-COV-2), comprising administering to a subject in need thereof an effective amount of a thiazolide agent to produce an effective concentration of tizoxanide in a plasma of the subject.

### FIGURES

FIG. 1 shows plots presenting a proportion of non-responders as a function of a time from administering a first dose of nitazoxanide (or placebo) to return to usual health for all coronavirus-infected subjects (left panel) and subjects with coronavirus a sole pathogen (right panel).

FIG. 2 shows plots presenting a proportion of non-responders as a function of a time from administering a first dose of nitazoxanide (or placebo) to return to usual health for all coronavirus-infected subjects (left panel) and subjects with coronavirus a sole pathogen (right panel).

FIG. 3 shows a plot presenting a proportion of non-responders as a function of a time from administering a first dose of nitazoxanide (or placebo) to a sustained response for coronavirus-infected subjects who passed the 4-question test. Sustained response is defined as: (i) reduction of total FLU-PRO symptom score from the prior day, (ii) subject reports overall symptoms are either “somewhat better” or “much better” than yesterday, (iii) no oral temperature ≥100.4° C. during last 24 hours, (iv) no subsequent increase in any mean domain or subdomain score except under background levels where such background levels are body/systemic ≤0.56, throat ≤0.67, eyes ≤0.67, gastrointestinal ≤2.0, head ≤2.0, nose ≤0.75, chest=0, cough ≤1.75).

FIG. 4 shows plots presenting a proportion of non-responders as a function of a time from administering a first dose of nitazoxanide (or placebo) to a sustained response (A) for all coronavirus-infected subjects who passed the 4-question test (B) for coronavirus-infected subjects who passed the 4-question test and reported a FLU-PRO® score for at least one respiratory symptom selected from a chest pain, cough, nose, throat and head being 2 or greater; (C) for coronavirus-infected subjects who passed the 4-question test and reported a FLU-PRO® score for at least one respiratory symptom selected from a chest pain, cough, nose, throat and head being 3 or greater.

FIG. 5 shows FLU-PRO® domain/subdomain structure. For purposes of analyzing FLU-PRO® data, the responses to FLU-PRO® questions are assigned scores of 0 to 4 with 4 being the most severe.

FIG. 6 shows subject disposition for the study of Example 5.

FIG. 7A-B show clinical recovery in subjects with mild COVID-19 illness. (A) Time to sustained response in subjects with mild COVID-19 illness; (B) time to return to usual health in subjects with mild COVID-19 illness.

### DETAILED DESCRIPTION

As used herein and in the claims, the singular forms “a,” “an,” and “the” include the plural reference unless the context clearly indicates otherwise. Throughout this specification, unless otherwise indicated, “comprise,” “comprises” and “comprising” are used inclusively rather than exclusively, so that a stated integer or group of integers may include one or more other non-stated integers or groups of integers. The term “or” is inclusive unless modified, for example, by “either.” Thus, unless context indicates otherwise, the word “or” means any one member of a particular list and also includes any combination of members of that list. Other than in the operating examples, or where otherwise indicated, all numbers expressing quantities of ingredients or reaction conditions used herein should be understood as modified in all instances by the term “about.”

Headings are provided for convenience only and are not to be construed to limit the invention in any way. Unless defined otherwise, all technical and scientific terms used herein have the same meaning as those commonly understood to one of ordinary skill in the art. The terminology used herein is for the purpose of describing particular embodiments only, and is not intended to limit the scope of the present invention, which is defined solely by the claims. In order that the present disclosure can be more readily understood, certain terms are first defined.

All numerical designations, e.g., pH, temperature, time, concentration, and molecular weight, including ranges, are approximations which are varied (+) or (−) by increments of 1, 5, or 10%. It is to be understood, although not always explicitly stated that all numerical designations are preceded by the term “about.” It also is to be understood, although not always explicitly stated, that the reagents described herein are merely exemplary and that equivalents of such are known in the art are set forth throughout the detailed description.

NTZ refers to nitazoxanide, also known as 2-(acetolyloxy)-N-(5-nitro-2-thiazolyl) benzamide, which is a compound having the following structure:

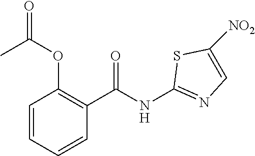 [SMILES: CC(=O)Oc1ccccc1C(=O)Nc1ncc([N+](=O)[O-])s1]

Tizoxanide is the active circulating metabolite of nitazoxanide. Tizoxanide has the following formula:

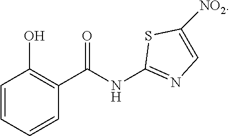 [SMILES: O=C(Nc1ncc([N+](=O)[O-])s1)c1ccccc1O]

Another metabolite of nitazoxanide is glucoronotizoxanide, which has the following formula:

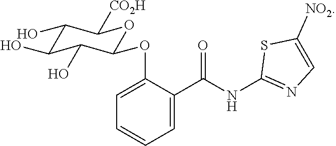 [SMILES: O=C(Nc1ncc([N+](=O)[O-])s1)c1ccccc1OC1OC(C(=O)O)C(O)C(O)C1O]

Nitazoxanide is approved in the United States for the treatment of diarrhea caused by Cryptosporidium parvum and Giardia lamblia.

Thiazolide compounds may be synthesized, for example, according to published procedures U.S. Pat. Nos. 3,950,351 and 6,020,353, PCTWO2006042195A1 and US2009/0036467A.

Pharmaceutical compositions containing nitazoxanide and its metabolite, tizoxanide, were originally developed and marketed for treating intestinal parasitic infections. Various applications of nitazoxanide, tizoxanide and other thiazolide compounds, such as RM-4848, are disclosed, for example, U.S. Pat. Nos. RE47,786, 10,383,855, 10,363,243, 10,358,428, 10,336,058, RE47,404, 10,100,023, RE46,724, 9,827,227, 9,820,975, 9,351,937, 9,345,690, 9,126,992, 9,107,913, 9,023,877, 8,895,752, 8,846,727, 8,772,502, 8,633,230,8,524,278, 8,124,632, 7,645,783, 7,550,493, 7,285,567, 6,117,894, 6,020,353, 5,968,961, 5,965,590, 5,935,591, 5,886,013, 5,859,038, 5,856,348 as well as in U.S. patent application publications Nos. 20200038377, 20190321338, 20190307730, 20190291404, 20190276417, 20190040026, 20180126722, 20180085353, 2018078533, 20170334868, 20170281603, 20160243087, 20160228415, 2015025768, 20140341850, 20140112888, 20140065215, 20120294831, 20120122939, 20120108592, 20120108591, 20100330173, 20100292274, 20100209505, 20090036467, 20080097106, 20080097106, 20080096941, 20070167504, 20070015803, 20060194853, 20060089396, 20050171169, each of which is incorporated herein by reference in its entirety. The present application also incorporates by reference in its entirety U.S. provisional application No. 63/155,481 filed Mar. 2, 2021 titled “TREATMENT OF VIRAL RESPIRATORY ILLNESS IN SELECTED PATIENT POPULATION.”

The present inventors discovered that administering to a subject, such as a human being, an effective amount of a thiazolide agent, which produces an effective concentration of tizoxanide in plasma of the subject upon the administering, may treat and/or prevent an illness caused by a virus belonging to the Coronaviridae family, such as a virus belonging to the Orthocoronavirinae subfamily. As used herein, the term “treating and/or preventing a illness caused by a virus” may include at least one of the following: inhibiting the replication of the virus, inhibiting viral transmission, preventing the virus from establishing itself in its host, ameliorating or alleviating the symptoms or progression of the illness caused by the virus. The treatment is considered therapeutic if there is at least one of a reduction in viral load, decrease in mortality and/or morbidity related with the illness, decrease in the progression of the illness or a shorter duration of the illness. In certain embodiments, “treating and/or preventing an illness caused by a virus” may include increased survival among subjects affected with the illness and treated with a thiazolide agent compared to subjects affected with the illness but not treated with the thiazolide agent, such as subjects receiving a placebo. In certain embodiments, “treating and/or preventing an illness caused by a virus” may include reduction of a viral load in a subject affected with the illness upon administering a thiazolide agent. Yet in some embodiments, “treating and/or preventing an illness caused by a virus” may include ameliorating or alleviating the symptoms or progression of the illness caused by the virus. In some embodiments, “treating and/preventing an illness caused by a virus” may include a statistically significant reduction of time to alleviation of symptoms in subjects affected with the illness upon administering a thiazolide agent compared to subjects affected with the illness but not treated with the thiazolide agent, such as subjects receiving a placebo. In some embodiments, “treating and/preventing an illness caused by a virus” may include a statistically significant reduction of time until return to usual health in subjects affected with the illness upon administering a thiazolide agent compared to subjects affected with the illness but not treated with the thiazolide agent, such as subjects receiving a placebo. In some embodiments, “treating and/preventing an illness caused by a virus” may include a statistically significant reduction of time until ability to perform all normal activities in subjects affected with the illness upon administering a thiazolide agent compared to subjects affected with the illness but not treated with the thiazolide agent, such as subjects receiving a placebo.

### Coronaviridae Viral Family

The family Coronaviridae includes 2 sub-families: the Orthocoronavirinae subfamily (also known as the Coronavirinae family) and the Letovirinae subfamily.

The Orthocoronavirinae subfamily includes 4 genera: the Alphacoronavirus genus; the Betacoronavirus genus; the Gammacoronavirus genus; and the Alphacoronavirus genus.

Genus Alphacoronavirus includes the following species: Transmissible gastroenteritis coronavirus (TGEV); Alphacoronavirus 1, which includes Canine coronavirus, Feline coronavirus, Transmissible gastroenteritis coronavirus; Human coronavirus 229E; Human coronavirus NL63; Miniopterus bat coronavirus 1; Miniopterus bat coronavirus HKU8; Porcine epidemic diarrhea virus; Rhinolophus bat coronavirus HKU2; and Scotophilus bat coronavirus 512.

Genus Betacoronavirus includes the following species: Murine coronavirus (MHV); Betacoronavirus 1, which includes Bovine Coronavirus, Human coronavirus OC43; Hedgehog coronavirus 1; Human coronavirus HKU1; Middle East respiratory syndrome-related coronavirus; Murine coronavirus; Pipistrellus bat coronavirus HKU5; Rousettus bat coronavirus HKU9; Severe acute respiratory syndrome-related coronavirus, which includes SARS-CoV, SARS-CoV-2; Tylonycteris bat coronavirus HKU4.

Genus Gamacoronavirus includes the following species: Avian coronavirus, Beluga whale coronavirus SW1.

Genus Deltacoronavirus includes the following species: Bulbul coronavirus HKU11, Porcine coronavirus HKU15.

### Illness

An illness caused by a virus belonging to the Coronaviridae family, such as a virus belonging to the Orthocoronavirinae subfamily, may be pronounced in one or more symptoms displayed by the subject, such as a human being, who tested positive for the virus.

The one or more symptoms may include an elevated temperature or a fever, which may be, for example, an orally measured temperature of no less than 37.3° C. or of no less than 37.4° C. or of no less than 37.5° C. or no less than 37.6° C. or no less than 37.7° C. or no less than 37.8° C. or no less than 37.9° C. or no less than 38.0° C. or no less than 38.1° C. or no less than 38.2° C. or no less than 38.3° C. or no less than 38.4° C. or no less than 38.5° C.

In some embodiments, the one or more symptoms of the illness may also include a) one or more respiratory symptoms, which may include one or more of cough, sore throat and nasal congestion; and/or one or more constitutional symptoms, which may include fatigue, headache, myalgia and feverishness.

In some embodiments, the subject may display one or more symptoms selected from the elevated temperature, one or more respiratory symptoms such as cough, sore throat and nasal congestion; and one or more constitutional symptoms, such as fatigue, headache, myalgia and feverishness.

In some embodiments, the subject may display two or more symptoms selected from the elevated temperature, one or more respiratory symptoms such as cough, sore throat and nasal congestion; and one or more constitutional symptoms, such as fatigue, headache, myalgia and feverishness.

In some embodiments, the subject may display three or more symptoms selected from the elevated temperature, one or more respiratory symptoms such as cough, sore throat and nasal congestion; and one or more constitutional symptoms, such as fatigue, headache, myalgia and feverishness.

In some embodiments, the subject may display four or more symptoms selected from the elevated temperature, one or more respiratory symptoms such as cough, sore throat and nasal congestion; and one or more constitutional symptoms, such as fatigue, headache, myalgia and feverishness.

In some embodiments, a virus belonging to the Coronaviridae family, such as a virus belonging to a virus belonging to the Orthocoronavirinae subfamily, may be a sole cause of the illness and its symptom(s). For example, in some embodiments, the subject may have a negative test result for any of the following pathogens: influenza A (non-specific as to subtype), influenza A/H1, A/H1N1 (2009), A/H3 subtypes, influenza B, respiratory syncytial virus A and B (RSV), parainfluenza 1, 2, 3 and 4, human metapneumovirus (hMPV), adenovirus (A-F), human rhinovirus/enterovirus, Chlamydophila pneumoniae, and Mycoplasma pneumoniae. Testing for any of these pathogens as well as testing for a virus belonging to the Coronaviridae family, such as a virus belonging to a virus belonging to the Orthocoronavirinae subfamily, may be performed by obtaining a sample of the subject, which may be, for example, a saliva sample or a nasopharyngeal swab, and running reverse transcription polymerase chain reaction (RT-PCR). An RT-PCR testing of a panel of respiratory pathogens is commercially available from, for example, Genmark, Carlsbad, California as the ePlex® Respiratory Pathogen Panel.

Yet in some embodiments, the illness in the subject may be caused one or more additional pathogens in addition to the virus belonging to the Coronaviridae family, such as a virus belonging to a virus belonging to the Orthocoronavirinae subfamily. Examples of such additional pathogens may include Influenza virus, such as Influenza A virus, and a virus belonging to the Enterovirus genus of the Picornaviridae family, which may be an enterovirus and/or a rhinovirus. For example, the illness and its symptoms may be caused by a combination of the virus belonging to the Coronaviridae family, such as a virus belonging to a virus belonging to the Orthocoronavirinae subfamily, and Influenza A virus when the subject test positive for both the virus belonging to the Coronaviridae family and Influenza A virus. The illness and its symptoms may be caused by a combination of the virus belonging to the Coronaviridae family, such as a virus belonging to a virus belonging to the Orthocoronavirinae subfamily, and a virus belonging to the Enterovirus genus of the Picornaviridae family, which may be an enterovirus and/or a rhinovirus, when the subject test positive for both the virus belonging to the Coronaviridae family and the virus belonging to the Enterovirus genus of the Picornaviridae family.

In some embodiments, the illness and its symptoms may be caused by a virus belonging to the Alphacoronavirus genus. In some embodiments, the virus belonging to the Alphacoronavirus genus may be a sole cause of the illness and its symptoms. Yet in some embodiments, the illness in the subject may be caused one or more additional pathogens, such as those discussed above, in addition to the virus belonging to the Alphacoronavirus genus.

In some embodiments, the illness and its symptoms may be caused by a virus belonging to the Betacoronavirus genus. In some embodiments, the virus belonging to the Betacoronavirus genus may be a sole cause of the illness and its symptoms. Yet in some embodiments, the illness in the subject may be caused one or more additional pathogens, such as those discussed above, in addition to the virus belonging to the Betacoronavirus genus.

In some embodiments, the illness and its symptoms may be caused by a virus selected from the group consisting of NL63 coronavirus, HKU1 coronavirus, 229E coronavirus, and OC43 coronavirus. In some embodiments, such virus may be a sole cause of the illness and its symptoms. Yet in some embodiments, the illness in the subject may be caused one or more additional pathogens, such as those discussed above, in addition to the virus selected from the group consisting of NL63 coronavirus, HKU1 coronavirus, 229E coronavirus, and OC43 coronavirus.

In some embodiments, the illness may be such that it has an average duration of symptom(s) in patients who are affected by the illness but remain untreated or are treated with a placebo, of at least 160 hours or at least 170 hours or at least 180 hours or at least 190 hours or at least 200 hours or at least 210 hours or at least 220 hours or at least 230 hours or at least 240 hours or at least 250 hours from an onset of the symptom(s).

In some embodiments, the illness may be such that an average time to return to usual health in patients who are affected by the illness but remain untreated or are treated with a placebo, is at least 160 hours or at least 170 hours or at least 180 hours or at least 190 hours or at least 200 hours or at least 210 hours or at least 220 hours or at least 230 hours or at least 240 hours or at least 250 hours from an onset of symptom(s) of the illness.

In some embodiments, the illness may be such that an average time to return to all normal activities in patients who are affected by the illness but remain untreated or are treated with a placebo, is at least 160 hours or at least 170 hours or at least 180 hours or at least 190 hours or at least 200 hours or at least 210 hours or at least 220 hours or at least 230 hours or at least 240 hours or at least 250 hours from an onset of symptom(s) of the illness.

### Patient

In some embodiments, a patient may be selected using the methodology disclosed in U.S. provisional application No. 63/155,481 filed Mar. 2, 2021 titled “TREATMENT OF VIRAL RESPIRATORY ILLNESS IN SELECTED PATIENT POPULATION” which is incorporated by reference in its entirety.

In some embodiments, a patient may be a human being, who tested positive to a virus belonging to the Coronaviridae family and who provided acceptable responses to the following four FLU-PRO questions (“four question test”) before being administered with a thiazolide agent, such as tizoxanide, a pharmaceutically acceptable salt of tizoxanide and/or a prodrug of tizoxanide, such as nitazoxanide:
(A) Have you returned to your usual health today?
Yes (unacceptable response)
No (acceptable response)
(B) How much did your symptoms interfere with your usual activities today?
Not at all (unacceptable response)
A little bit (acceptable response)
Somewhat (acceptable response)
Quite a bit (acceptable response)
Very much (acceptable response)
(C) Overall how severe were your flu symptoms today?
No flu symptoms today (unacceptable response)
Mild (unacceptable response)
Moderate (acceptable response)
Severe (acceptable response)
Very severe (acceptable response)
(D) Overall how were your flu symptoms today compared to yesterday?
Much better (unacceptable response)
Somewhat better (unacceptable response)
A little better (unacceptable response)
About the same (acceptable response)
A little worse (acceptable response)
Somewhat worse (acceptable response)
Much worse (acceptable response)

In some embodiments, a patient may be an immunologically naïve patient, i.e. a patient without a previous exposure to a virus belonging to the Coronaviridae virus for which the patient is tested positive.

In some embodiments, a patient may be free of antibodies to a virus belonging to the Coronaviridae family for which the patient is tested positive. For example, a sample of a body fluid, such as a blood sample, may be collected from the patient and send to a laboratory for testing to detect antibodies to the virus. In some embodiments, a patient may be free of antibodies to any human virus belonging to the Coronaviridae family, including the virus for which the patient is tested positive and other human viruses, which belong to the Coronaviridae family but for which the patient is not tested positive. In some embodiments, a patient may be free of antibodies to any virus belonging to the Coronaviridae family, including the virus for which the patient is tested positive and other viruses, which belong to the Coronaviridae family but for which the patient is not tested positive.

In some embodiments, the patient may be a patient with an elevated temperature or a fever, which may be, for example, an orally measured temperature of no less than 37.3° C. or of no less than 37.4° C. or of no less than 37.5° C. or no less than 37.6° C. or no less than 37.7° C. or no less than 37.8° C. or no less than 37.9° C. or no less than 38.0° C. or no less than 38.1° C. or no less than 38.2° C. or no less than 38.3° C. or no less than 38.4° C. or no less than 38.5° C. In some embodiments, such patient may further satisfy one or both of the following criteria (a) provide an acceptable response to each of the four questions of the four-question test; (b) be free of antibodies to a virus belonging to the Coronaviridae family for which the patient is tested positive and/or be free of antibodies to any human virus belonging to the Coronaviridae family and/or be free of antibodies to any virus belonging to the Coronaviridae family.

In some embodiments, the patient may be a patient with at least one symptom, which may include head symptoms, such as headache, head congestion (the head subdomain); throat symptoms, such as difficulty swallowing, sore or painful throat, scratchy or itchy throat (the throat domain); nose symptoms, such as stuffy/congested nose, runny/dripping nose, sinus pressure and sneezing (the nose domain); chest symptoms, such as trouble breathing, chest tightness and chest congestion (the chest subdomain); cough symptoms, such as coughing, dry or hacking cough, coughed up mucus or phlegm, wet or loose cough (the cough subdomain), and the mean score for at least one of these five domains or subdomains is equal or more than 2.0 or equal or more than 3.0 as determined using the FLU-PRO® questionnaire. In some embodiments, such patient may further satisfy at least one, at least two or at least three of the following criteria (a) provide an acceptable response to each of the four questions of the four question test; (b) be free of antibodies to a virus belonging to the Coronaviridae family for which the patient is tested positive and/or be free of antibodies to any human virus belonging to the Coronaviridae family and/or be free of antibodies to any virus belonging to the Coronaviridae family; (c) have an elevated temperature or a fever as defined above.

In some embodiments, the patient may be a patient with at least one symptom listed in the head, throat, nose, chest or cough domains/subdomains of FLU-PRO®, and the mean score for at least two of the five domains or subdomains is equal or more than 2.0 or equal or more than 3.0. In some embodiments, such patient may further satisfy at least one, at least two or at least three of the following criteria (a) provide an acceptable response to each of the four questions of the four-question test; (b) be free of antibodies to a virus belonging to the Coronaviridae family for which the patient is tested positive and/or be free of antibodies to any human virus belonging to the Coronaviridae family and/or be free of antibodies to any virus belonging to the Coronaviridae family; (c) have an elevated temperature or a fever as defined above.

In some embodiments, the patient may be a patient with at least one symptom listed in the head, throat, nose, chest or cough domains/subdomains of FLU-PRO®, the mean score for at least one of these five domains or subdomains is equal or more than 2.0 or equal or more than 3.0, and a resting pulse rate of equal or more than 90 beats per minute (bpm) or equal or more than 92 bpm or equal or more than 95 bpm or equal or more than 98 bpm or equal or more 100 bpm. In some embodiments, such patient may further satisfy at least one, at least two or at least three of the following criteria (a) provide an acceptable response to each of the four questions of the four-question test; (b) be free of antibodies to a virus belonging to the Coronaviridae family for which the patient is tested positive and/or be free of antibodies to any human virus belonging to the Coronaviridae family and/or be free of antibodies to any virus belonging to the Coronaviridae family; (c) have an elevated temperature or a fever as defined above.

In some embodiments, the patient may be a patient with at least one symptom listed in the head, throat, nose, chest or cough domains/subdomains of FLU-PRO®, the mean score for at least one of these five domains or subdomains is equal or more than 2.0 or equal or more than 3.0, and a resting respiratory rate of equal or more 16 breaths per minute (bpm) or equal or more 18 bpm or equal or more 20 bpm or equal or more 21 bpm or equal or more 22 bpm. In some embodiments, such patient may further satisfy at least one, at least two or at least three of the following criteria (a) provide an acceptable response to each of the four questions of the four-question test; (b) be free of antibodies to a virus belonging to the Coronaviridae family for which the patient is tested positive and/or be free of antibodies to any human virus belonging to the Coronaviridae family and/or be free of antibodies to any virus belonging to the Coronaviridae family; (c) have an elevated temperature or a fever as defined above.

Yet in some embodiments, the patient may be a patient with at least one symptom listed in the head, throat, nose, chest or cough domains/subdomains of FLU-PRO®, the mean score for at least two of the five domains or subdomains is equal or more than 2.0 or equal or more than 3.0, a resting pulse rate of less than 90 bpm and a resting respiratory rate of less than 20 breaths per minute. In some embodiments, such patient may further satisfy at least one, at least two or at least three of the following criteria (a) provide an acceptable response to each of the four questions of the four-question test; (b) be free of antibodies to a virus belonging to the Coronaviridae family for which the patient is tested positive and/or be free of antibodies to any human virus belonging to the Coronaviridae family and/or be free of antibodies to any virus belonging to the Coronaviridae family; (c) have an elevated temperature or a fever as defined above.

The patient in this section may be a patient who tested positive to a virus belonging to the Coronaviridae family, which may be for example, a virus belonging to the Alphacoronavirus genus; a virus belonging to the Betacoronavirus genus; the virus selected from the group consisting of NL63 coronavirus, HKU1 coronavirus, 229E coronavirus, and OC43 coronavirus; or a severe acute respiratory syndrome-related coronavirus (SARS), which may be SARS-CoV or SARS-CoV-2.

### Thiazolide Agent

The thiazolide agent may comprise tizoxanide, a pharmaceutically acceptable salt of tizoxanide and/or a prodrug of tizoxanide.

In some embodiments, a prodrug of tizoxanide may be nitazoxanide.

Yet in some embodiments, a prodrug of tizoxanide may be a prodrug of tizoxanide disclosed in WO2016/077420 and U.S. Pat. Nos. 10,100,023; 10,358,428; 10,577,337, each of which is incorporated by reference in its entirety. Such prodrug may have a formula:

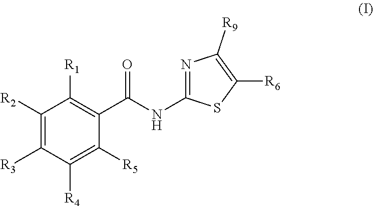

where R₆ is NO₂; each of R₂, R₃, R₄, R₅ and R₉ is hydrogen; R₁ is as defined in, for example, WO2016/077420. Non-limiting examples of such prodrugs include RM-5061, which has the following formula:

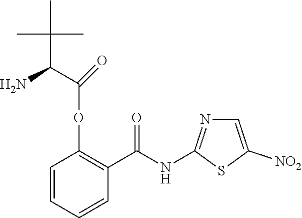 [SMILES: CC(C)(C)[C@H](N)C(=O)Oc1ccccc1C(=O)Nc1ncc([N+](=O)[O-])s1]

and RM-5066 as defined, for example, in WO2016/077420.

In some embodiments, a pharmaceutically acceptable salt of tizoxanide may be a salt of tizoxanide disclosed in U.S. provisional application No. 63/054,072 filed Jul. 20, 2020 titled “Salts of Tizoxanide” or in corresponding PCT application No. PCT/US2021/042196 filed Jul. 19, 2021 both of which are incorporated by reference in its entirety. Such salts include amine containing salts of tizoxanide such as a salt of tizoxanide formed with a liquid amine containing base, such as ammonia, methylamine, diethylamine, ethanolamine, dicyclohexylamine, N-methylmorpholine, ammonium, tetramethylammonium, tetraethylammonium, methylamine, dimethylamine, trimethylamine, triethylamine, diethylamine, ethylamine, tributylamine, pyridine, N,N-dimethylaniline, N-methylpiperidine, N-methylmorpholine, dicyclohexylamine, procaine, dibenzylamine, N,N-dibenzylphenethylamine, 1-ephenamine, and N,N′-dibenzylethylenediamine, ethylenediamine, ethanolamine, diethanolamine, piperidine.

### Compositions

The thiazolide agent, such as tizoxanide, a pharmaceutically acceptable salt of tizoxanide and/or a prodrug of tizoxanide, which may be nitazoxanide, may be administered as a part of a pharmaceutical composition. The pharmaceutical composition may include in addition to the thiazolide agent may include a carrier, such as a pharmaceutically acceptable carrier. The term “carrier” may be used in its broadest sense. For example, the term “carrier” refers to any carriers, diluents, excipients, wetting agents, buffering agents, suspending agents, lubricating agents, adjuvants, vehicles, delivery systems, emulsifiers, disintegrants, absorbents, preservatives, surfactants, colorants, flavorants, and sweeteners. In some embodiments, the carrier may be a pharmaceutically acceptable carrier, a term narrower than carrier, because the term pharmaceutically acceptable carrier” means a non-toxic that would be suitable for use in a pharmaceutical composition. Actual dosage levels of the thiazolide agent in the pharmaceutical composition may vary so as to administer an amount of the thiazolide agent is effective to provide an effective concentration of tizoxanide in plasma of the subject and therefore to achieve the desired therapeutic response for a particular patient.

The selected dose level may depend on the activity of the specific thiazolide agent, the route of administration, the severity of the condition being treated, and the condition and prior medical history of the patient being treated. However, it is within the skill of the art to start doses of the thiazolide agent(s) at levels lower than required to achieve the desired therapeutic effect and to gradually increase the dosage until the desired effect is achieved. If desired, the effective daily dose may be divided into multiple doses for purposes of administration, for example, two to four doses per day. It will be understood, however, that the specific dose level for any particular patient may depend on a variety of factors, including the body weight, general health, diet, time and route of administration and combination with other therapeutic agents and the severity of the condition or disease being treated.

The pharmaceutical composition may be administered systemically, for example, in an oral formulation, such as a solid oral formulation. For example, it may be in the physical form of a powder, tablet, capsule, lozenge, gel, solution, suspension, syrup, or the like. In some embodiments, the pharmaceutical composition may be in a form of a formulation disclosed in U.S. Pat. Nos. 8,524,278 and 9,351,937. Such formulation may, for example, be an extended release formulation, which includes a controlled release portion, which contains a first amount of a thiazolide agent, which may be, for example, nitazoxanide and/or tizoxanide, and an immediate release portion, which contains a second amount of a thiazolide agent, which may be, for example, nitazoxanide and/or tizoxanide. Together the first amount and the second amount may provide an effective amount of the thiazolide agent, which may provide an effective concentration of tizoxanide in plasma of the subject, such as a human being. These compositions may be administered in a single dose or in multiple doses which are administered at different times.

In some embodiments, the total amount of the thiazolide agent, such as nitazoxanide and/or tizoxanide, may be from about 20% to about 95% or from about 30% to about 90% or from about 35% to about 85% or from about 60% to about 75% by weight of the composition. The composition may be formulated for immediate release, controlled release or sustained release. The compositions may contain one or more additional pharmaceutically acceptable additives or excipients. These excipients are therapeutically inert ingredients that are well known and appreciated in the art. As used herein, the term “inert ingredient” may refer to those therapeutically inert ingredients that are well known in the art of pharmaceutical manufacturing, which can be used singly or in various combinations, and include, for example, diluents, disintegrants, binders, suspending agents, glidants, lubricants, fillers, coating agents, solubilizing agent, sweetening agents, coloring agents, flavoring agents, and antioxidants. See, for example, Remington: The Science and Practice of Pharmacy 1995, edited by E. W. Martin, Mack Publishing Company, 19th edition, Easton, Pa.

Examples of diluents or fillers include, but are not limited to, starch, lactose, xylitol, sorbitol, confectioner's sugar, compressible sugar, dextrates, dextrin, dextrose, fructose, lactitol, mannitol, sucrose, talc, microcrystalline cellulose, calcium carbonate, calcium phosphate dibasic or tribasic, dicalcium phosphate dehydrate, calcium sulfate, and the like. The amount of diluents or fillers may be in a range between about 2% to about 15% by weight of the entire composition.

Examples of disintegrants include, but are not limited to, alginic acid, methacrylic acid DVB, cross-linked PVP, microcrystalline cellulose, sodium croscarmellose, crospovidone, polacrilin potassium, sodium starch glycolate, starch, including corn or maize starch, pregelatinized starch and the like. Disintegrant(s) typically represent about 2% to about 15% by weight of the entire composition.

Examples of binders include, but are not limited to, starches such as potato starch, wheat starch, corn starch; microcrystalline cellulose; celluloses such as hydroxypropyl cellulose, hydroxyethyl cellulose, hydroxypropylmethyl cellulose (HPMC), ethyl cellulose, sodium carboxy methyl cellulose; natural gums like acacia, alginic acid, guar gum; liquid glucose, dextrin, povidone, syrup, polyethylene oxide, polyvinyl pyrrolidone, poly-N-vinyl amide, polyethylene glycol, gelatin, poly propylene glycol, tragacanth, and the like. The amount of binder(s) is about 0.2% to about 14% by weight of the entire composition.

Examples of glidants include, but are not limited to, silicon dioxide, colloidal anhydrous silica, magnesium trisilicate, tribasic calcium phosphate, calcium silicate, magnesium silicate, colloidal silicon dioxide, powdered cellulose, starch, talc, and the like. The amount of glidant(s) is about 0.01% to about 0.3% by weight of the entire composition.

Examples of lubricants include, but are not limited to, magnesium stearate, aluminum stearate, calcium stearate, zinc stearate, stearic acid, polyethylene glycol, glyceryl behenate, mineral oil, sodium stearyl fumarate, talc, hydrogenated vegetable oil and the like. The amount of lubricant(s) is about 0.2% to about 1.0% by weight of the entire composition.

The compositions may contain a binder that is a low-viscosity polymer. Examples of low-viscosity polymers include, but are not limited to, low-viscosity hydroxypropyl methylcellulose polymers such as those sold by Dow Chemical under the tradename “MethoceL™” (e.g., Methocel E50LV™, Methocel K100LVR™, and Methocel F50LVR™) and low-viscosity hydroxyethylcellulose polymers. The low-viscosity polymer is typically present at about 10% to about 20%, or about 10% to about 15%, or preferably about 12%, of the total weight of the entire composition, or, in those embodiments having controlled release and immediate release portions, the low-viscosity polymer in the controlled release portion is typically present at about 15% to about 20%, preferably about 18%, of the weight of the controlled release portion.

The compositions may further comprise a coating material. The coating material is typically present as an outer layer on the dosage form that completely covers the formulation. For example, in some embodiments, the dosage form is an oral tablet in which the controlled release portion forms a first layer of the tablet and the immediate release portion forms a second layer that is deposited on top of the first layer to form a core tablet. In such embodiments, e.g., the coating material can be in the form of an outer coating layer that is deposited on top of the core tablet. The coating material typically is about 1% to about 5% by weight of the composition, and may comprise hydroxypropylmethylcellulose and/or polyethylene glycol, and one or more excipients selected from the group comprising coating agents, opacifiers, taste-masking agents, fillers, polishing agents, coloring agents, antitacking agents and the like. Examples of film-coating substances and methods for using such coating substances are well known to those of skill in the art.

### Administration

A thiazolide agent, such as nitazoxanide and/or tizoxanide, may be administered for a length of time suitable to effectively treat an illness caused by a virus belonging to the Coroviridae family, such as a virus belonging the Orthocoronavirinae subfamily. A number of appropriate dosages and regimen may be used for the compositions. In some embodiments, administration may be carried out over a period of about 3 days to about 104 weeks. In some embodiments, administration may be carried out over a period longer than 104 weeks and may even be carried out indefinitely. Appropriate regimens may be determined by a physician.

In some embodiments, administering of a thiazolide agent, such as nitazoxanide and/or tizoxanide, may start within 24 hours or within 30 hours or within 35 hours or within 40 hours or within 45 hours or within 50 hours or within 60 hours or within 72 hours or within 96 hours from an onset in the subject, such as a human being, of at least one symptom of the illness caused by a virus belonging to the Coroviridae family, such as a virus belonging the Orthocoronavirinae subfamily. For example, administering of a thiazolide agent, such as nitazoxanide and/or tizoxanide, may start within 24 hours or within 30 hours or within 35 hours or within 40 hours or within 45 hours or within 50 hours or within 60 hours or within 72 hours or with 96 hours from an onset in the subject, such as a human being, one or more symptoms selected from an elevated temperature or a fever (such as an orally measured temperature of no less than 37.3° C. or of no less than 37.4° C. or of no less than 37.5° C. or no less than 37.6° C. or no less than 37.7° C. or no less than 37.8° C. or no less than 37.9° C. or no less than 38.0° C. or no less than 38.1° C. or no less than 38.2° C. or no less than 38.3° C. or no less than 38.4° C. or no less than 38.5° C.); one or more respiratory symptoms, such as cough, sore throat and nasal congestion; and one or more constitutional symptoms, such as fatigue, headache, myalgia and feverishness.

In some embodiments, a daily dose of a thiazolide agent, such as nitazoxanide and/or tizoxanide, administered to a human may be from 100 mg to 1300 mg or from 200 mg to 1200 mg or from 250 mg to 1100 mg or from 300 mg to 1000 mg or any dose value or subrange within these ranges. Exemplary dosage values include 100 mg, 200 mg, 300 mg, 400 mg, 500 mg, 600 mg, 700 mg or 800 mg.

In some embodiments, a thiazolide agent, such as nitazoxanide and/or tizoxanide, may be administered at least for 2 days or at least for 3 days or at least for 4 days or at least for 5 days or at least for 6 days. In some embodiments, a thiazolide agent, such as nitazoxanide and/or tizoxanide, may be administered for a period from 2 to 14 days or from 3 to 10 days or from 4 to 7 days or any value or subrange within these ranges. In certain embodiments, a thiazolide agent, such as nitazoxanide and/or tizoxanide, may be administered for 5 days. The dose of the thiazolide agent, such as nitazoxanide and/or tizoxanide, may be from 300 mg to 900 mg or from 400 mg to 800 mg or from 500 mg to 700 mg or any dose value or subrange within these ranges. Exemplary dosage values include 300 mg, 400 mg, 500 mg, 600 mg, 700 mg or 800 mg. The thiazolide agent, such as nitazoxanide and/or tizoxanide, may be administered once, twice or thrice daily. In certain cases, 600 mg of nitazoxanide and/or tizoxanide may be administered twice daily.

### Treatment

In some embodiments, administering a thiazolide agent, such as nitazoxanide and/or tizoxanide may result in a statistically significant reduction of time to alleviation of symptoms in subjects affected with the illness upon administering the thiazolide agent compared to subjects affected with the illness but not treated with the thiazolide agent, such as subjects receiving a placebo. For example, a duration of symptom(s) of the illness may be reduced by at least 10 hours or by at least 15 hours or by at least 20 hours or by at least 25 hours or by at least 30 hours or by at least 35 hours or by at least 40 hours or by at least 45 hours or by at least 50 hours or by at least 55 hours or by at least 60 hours in subjects affected with the illness upon administering the thiazolide agent compared to subjects affected with the illness but not treated with the thiazolide agent, such as subjects receiving a placebo. In some embodiments, a duration of symptom(s) of the illness may be reduced by at least 10 hours or by at least 15 hours or by at least 20 hours or by at least 25 hours or by at least 30 hours or by at least 35 hours or by at least 40 hours or by at least 45 hours or by at least 50 hours or by at least 55 hours or by at least 60 hours in subjects affected with the illness upon administering the thiazolide agent compared to subjects affected with the illness but not treated with the thiazolide agent, such as subjects receiving a placebo, when the illness has an average duration of symptom(s) in subject who are affected by the illness but remain untreated or are treated with a placebo, of at least 160 hours or at least 170 hours or at least 180 hours or at least 190 hours or at least 200 hours or at least 210 hours or at least 220 hours or at least 230 hours or at least 240 hours or at least 250 hours from an onset of the symptom(s).

In some embodiments, administering a thiazolide agent, such as nitazoxanide and/or tizoxanide may result in a statistically significant reduction of time until return to usual health in subjects affected with the illness upon administering a thiazolide agent, such as nitazoxanide and/or tizoxanide, compared to subjects affected with the illness but not treated with the thiazolide agent, such as subjects receiving a placebo. For example, a time from an onset of symptom(s) of the illness until return to usual health may be reduced by at least 10 hours or by at least 15 hours or by at least 20 hours or by at least 25 hours or by at least 30 hours or by at least 35 hours or by at least 40 hours or by at least 45 hours or by at least 50 hours or by at least 55 hours or by at least 60 hours in subjects affected with the illness upon administering a thiazolide agent, such as nitazoxanide and/or tizoxanide, compared to subjects affected with the illness but not treated with the thiazolide agent, such as subjects receiving a placebo. In some embodiments, a time from an onset of symptom(s) of the illness until return to usual health may be reduced by at least 10 hours or by at least 15 hours or by at least 20 hours or by at least 25 hours or by at least 30 hours or by at least 35 hours or by at least 40 hours or by at least 45 hours or by at least 50 hours or by at least 55 hours or by at least 60 hours in subjects affected with the illness upon administering a thiazolide agent, such as nitazoxanide and/or tizoxanide, compared to subjects affected with the illness but not treated with the thiazolide agent, such as subjects receiving a placebo when the illness has an average time to return to usual health in patients who are affected by the illness but remain untreated or are treated with a placebo, of at least 160 hours or at least 170 hours or at least 180 hours or at least 190 hours or at least 200 hours or at least 210 hours or at least 220 hours or at least 230 hours or at least 240 hours or at least 250 hours from an onset of symptom(s) of the illness.

In some embodiments, administering a thiazolide agent, such as nitazoxanide and/or tizoxanide may result in a statistically significant reduction of time until ability to perform all normal activities in subjects affected with the illness upon administering a thiazolide agent, such as nitazoxanide and/or tizoxanide, compared to subjects affected with the illness but not treated with the thiazolide agent, such as subjects receiving a placebo. For example, a time from an onset of symptom(s) of the illness until ability to perform all normal activities may be reduced by at least 10 hours or by at least 15 hours or by at least 20 hours or by at least 25 hours or by at least 30 hours or by at least 35 hours or by at least 40 hours or by at least 45 hours or by at least 50 hours or by at least 55 hours or by at least 60 hours in subjects affected with the illness upon administering a thiazolide agent, such as nitazoxanide and/or tizoxanide, compared to subjects affected with the illness but not treated with the thiazolide agent, such as subjects receiving a placebo. In some embodiments, a time from an onset of symptom(s) of the illness until ability to perform all normal activities may be reduced by at least 10 hours or by at least 15 hours or by at least 20 hours or by at least 25 hours or by at least 30 hours or by at least 35 hours or by at least 40 hours or by at least 45 hours or by at least 50 hours or by at least 55 hours or by at least 60 hours in subjects affected with the illness upon administering a thiazolide agent, such as nitazoxanide and/or tizoxanide, compared to subjects affected with the illness but not treated with the thiazolide agent, such as subjects receiving a placebo when the illness has an average time until ability to perform all normal activities in patients who are affected by the illness but remain untreated or are treated with a placebo, of at least 160 hours or at least 170 hours or at least 180 hours or at least 190 hours or at least 200 hours or at least 210 hours or at least 220 hours or at least 230 hours or at least 240 hours or at least 250 hours from an onset of symptom(s) of the illness.

A time until return to usual health and a time until ability to perform all normal activities may be evaluated using a patient-reported symptom scale measure, such as FLU-PRO® from Evidera or a similar measure. The FLU-PRO® measure and similar measures are disclosed, for example, in Powers J H, et al. BMC Infect Dis 2015; 16:1; Powers J H, et al. Value Health 2017; 21:210-18; Powers J H, et al. PLoS One 2018; 13:e0194180, Osborne R H, et al. J Outcomes Res 2000; 4:15-30, each of which is incorporated herein by references in its entirety.

### Treatment of a Mild or Moderate Illness Caused by Severe Acute Respiratory Syndrome Coronavirus

In some embodiments, administering a thiazolide agent, such as nitazoxanide and/or tizoxanide, may be used for treating a mild or moderate illness caused by severe acute respiratory syndrome coronavirus, such as SARS-COV-1 or SARS-COV-2.

In some embodiments, a patient with such mild or moderate illness may be a patient with at least one symptom listed in the head, throat, nose, chest or cough domains/subdomains of FLU-PRO®, and the mean score for at least two of the five domains or subdomains is equal or more than 2.0 or equal or more than 3.0. In some embodiments, such patient may further satisfy at least one, at least two or at least three of the following criteria (a) provide an acceptable response to each of the four questions of the four-question test; (b) be free of antibodies to severe acute respiratory syndrome coronavirus, such as SARS-COV-1 or SARS-COV-2, for which the patient is tested positive and/or be free of antibodies to any human virus belonging to the Coronaviridae family and/or be free of antibodies to any virus belonging to the Coronaviridae family; (c) have an elevated temperature or a fever as defined above. Domains/subdomains of FLU-PRO® are explained above as well as in U.S. provisional application No. 63/155,481 filed Mar. 2, 2021 titled “TREATMENT OF VIRAL RESPIRATORY ILLNESS IN SELECTED PATIENT POPULATION” which is incorporated herein by reference in its entirety.

In some embodiments, the patient with the mild or moderate illness may be a patient with at least one symptom listed in the head, throat, nose, chest or cough domains/subdomains of FLU-PRO®, the mean score for at least one of these five domains or subdomains is equal or more than 2.0 or equal or more than 3.0, and a resting pulse rate of equal or more than 90 beats per minute (bpm) or equal or more than 92 bpm or equal or more than 95 bpm or equal or more than 98 bpm or equal or more 100 bpm. In some embodiments, such patient may further satisfy at least one, at least two or at least three of the following criteria (a) provide an acceptable response to each of the four questions of the four-question test; (b) be free of antibodies to severe acute respiratory syndrome coronavirus, such as SARS-COV-1 or SARS-COV-2 for which the patient is tested positive and/or be free of antibodies to any human virus belonging to the Coronaviridae family and/or be free of antibodies to any virus belonging to the Coronaviridae family; (c) have an elevated temperature or a fever as defined above.

In some embodiments, the patient with the mild or moderate illness may be a patient with at least one symptom listed in the head, throat, nose, chest or cough domains/subdomains of FLU-PRO®, the mean score for at least one of these five domains or subdomains is equal or more than 2.0 or equal or more than 3.0, and a resting respiratory rate of equal or more 16 breaths per minute (bpm) or equal or more 18 bpm or equal or more 20 bpm or equal or more 21 bpm or equal or more 22 bpm. In some embodiments, such patient may further satisfy at least one, at least two or at least three of the following criteria (a) provide an acceptable response to each of the four questions of the four-question test; (b) be free of antibodies to severe acute respiratory syndrome coronavirus, such as SARS-COV-1 or SARS-COV-2, for which the patient is tested positive and/or be free of antibodies to any human virus belonging to the Coronaviridae family and/or be free of antibodies to any virus belonging to the Coronaviridae family; (c) have an elevated temperature or a fever as defined above.

Yet in some embodiments, the patient with the mild or moderate illness may be a patient with at least one symptom listed in the head, throat, nose, chest or cough domains/subdomains of FLU-PRO®, the mean score for at least two of the five domains or subdomains is equal or more than 2.0 or equal or more than 3.0, a resting pulse rate of less than 90 bpm and a resting respiratory rate of less than 20 breaths per minute. In some embodiments, such patient may further satisfy at least one, at least two or at least three of the following criteria (a) provide an acceptable response to each of the four questions of the four-question test; (b) be free of antibodies to severe acute respiratory syndrome coronavirus, such as SARS-COV-1 or SARS-COV-2, for which the patient is tested positive and/or be free of antibodies to any human virus belonging to the Coronaviridae family and/or be free of antibodies to any virus belonging to the Coronaviridae family; (c) have an elevated temperature or a fever as defined above.

Patients with the mild or moderate illness may exclude (a) patients with a severe illness caused by the severe acute respiratory syndrome coronavirus, such as SARS-COV-1 or SARS-COV-2, such as patients having one or more of the following indications: shortness of breath at rest, a resting pulse rate ≥125 beats per minute, a resting respiratory rate ≥30 breaths per minute, or oxygen saturation (SpO₂)≤93% on room air at sea level; (b) patients previously infected with the severe acute respiratory syndrome coronavirus, such as SARS-COV-1 or SARS-COV-2; (c) immunodeficient patients.

In some embodiments, a patient with the mild or moderate illness may be at least 12 years of age. Yet in some embodiments, a patient with the mild or moderate illness may be younger than 12 years of age.

Administering of a thiazolide agent, such as nitazoxanide and/or tizoxanide, to a patient, such as a human being, with the mild or moderate illness may start within 24 hours or within 30 hours or within 36 hours or within 42 hours or within 48 hours or within 54 hours or within 60 hours or within 66 hours, or within 72 hours or within 96 hours from an onset in the patient, of at least one symptom of the illness.

Administering a thiazolide agent, such as nitazoxanide and/or tizoxanide, to a patient population with a mild or moderate illness caused by a severe acute respiratory syndrome coronavirus, such as SARS-COV-1 or SARS-COV-2 may result in a statistically significant reduction of a progression to a severe illness caused by the severe acute respiratory syndrome coronavirus, such as SARS-COV-1 or SARS-COV-2 compared to an otherwise identical population with the mild or moderate illness but for whom a placebo was administered instead of the thiazolide compound. For example, a rate of progression to the severe illness may be reduced in the patient population that received the thiazolide compound compared to the patient population that remained untreated and/or received the placebo instead of the thiazolide compound by at least 20% or at least 25% or at least 30% or at least 35% or at least 40% or at least 45% or at least 50% or at least 55% or at least 60% or at least 65% or at least 70% or at least 75% or at least 80% or at least 85%.

Administering a thiazolide agent, such as nitazoxanide and/or tizoxanide, to a patient population with a mild or moderate illness caused by a severe acute respiratory syndrome coronavirus, such as SARS-COV-1 or SARS-COV-2 may result in a statistically significant reduction in a hospitalization rate due to the progression of the illness compared to that in an otherwise identical population with the mild or moderate illness but for whom a placebo was administered instead of the thiazolide compound. For example, a hospitalization rate in the patient population that received the thiazolide compound compared to that in the patient population that remained untreated and/or received the placebo instead of the thiazolide compound may be reduced by at least 20% or at least 25% or at least 30% or at least 35% or at least 40% or at least 45% or at least 50% or at least 55% or at least 60% or at least 65% or at least 70% or at least 75% or at least 80%.

Administering a thiazolide agent, such as nitazoxanide and/or tizoxanide, to a patient population with a mild or moderate illness caused by a severe acute respiratory syndrome coronavirus, such as SARS-COV-1 or SARS-COV-2 may result in a statistically significant reduction of a duration of symptoms of the illness compared to that in compared to that in an otherwise identical population with the mild or moderate illness but for whom a placebo was administered instead of the thiazolide compound. For example, a duration of symptom(s) of the illness may be reduced by at least 10 hours or by at least 15 hours or by at least 20 hours or by at least 25 hours or by at least 30 hours or by at least 35 hours or by at least 40 hours or by at least 45 hours or by at least 50 hours or by at least 55 hours or by at least 60 hours the patient population that received the thiazolide compound compared to that in the patient population that remained untreated and/or received the placebo instead of the thiazolide compound.

Administering a thiazolide agent, such as nitazoxanide and/or tizoxanide, to a patient population with a mild or moderate illness caused by a severe acute respiratory syndrome coronavirus, such as SARS-COV-1 or SARS-COV-2 may result in a statistically significant reduction of an average time of return to usual health compared to that in compared to that in an otherwise identical population with the mild or moderate illness but for whom a placebo was administered instead of the thiazolide compound. For example, an average time of return to usual health may be reduced by at least 10 hours or by at least 15 hours or by at least 20 hours or by at least 25 hours or by at least 30 hours or by at least 35 hours or by at least 40 hours or by at least 45 hours or by at least 50 hours or by at least 55 hours or by at least 60 hours the patient population that received the thiazolide compound compared to that in the patient population that remained untreated and/or received the placebo instead of the thiazolide compound.

### Prevention

In some embodiments, a thiazolide agent, such as tizoxanide, a pharmaceutically acceptable salt of tizoxanide and/or a prodrug of tizoxanide, such as nitazoxanide or RM-5161, may be used to prevent a viral respiratory illness in a subject, such as a human being, who may be have been exposed to the viral respiratory illness but does not display any symptom for the viral respiratory illness. For example, the subject may be not displaying any of the symptoms, such as fever, upper respiratory symptoms, such as nasal congestion/rhinorrhea (which may include runny or dripping nose, congested or stuffy nose, head congestion, sinus pressure); sore throat (e.g. sore or painful throat); lower respiratory symptoms, such cough (e.g. coughing, chest congestion, chest tightness, dry or hacking cough, wet or loose cough); dyspnea (e.g. shortness of breath); sputum (e.g. coughing up sputum or phlegm); wheezing; systemic symptoms, such as myalgia or arthralgias (e.g. body aches or pains); fatigue (e.g. weak or tired, sleeping more than usual); headache; decreased appetite (e.g. lack of appetite, did not feel like eating); feverishness (e.g. felt hot, chills or shivering, felt cold, sweating). The subject may have been suspected to being exposed to the viral respiratory illness. For example, the subject could have been a member of a restricted population, such as for example, a population of a nursing home or a long-term care facility, or a population of a cruise ship, in which one or more other members of the population have been infected to the viral respiratory illness. The subject could have been a medical professional or a first responder in close contact with a subject infected by the viral respiratory illness.

In some embodiments, the viral respiratory illness may be an illness caused by one or more viral pathogens selected from adenovirus, coronavirus, such as a human coronavirus, metapneumovirus, such as human metapneumovirus, enterovirus and/or rhinovirus, influenza, such as Influenza A or Influenza B, parainfluenza, and Respiratory Syncytial Virus (RSV). In some embodiments, the viral respiratory illness may be an illness caused by one or more viral pathogens selected from adenovirus, coronavirus, such as a human coronavirus, metapneumovirus, such as human metapneumovirus, enterovirus and/or rhinovirus, parainfluenza, and Respiratory Syncytial Virus (RSV).

In some embodiments, the viral respiratory illness may be caused by a virus belonging to the Coronaviridae family, such as a virus belonging to a virus belonging to the Orthocoronavirinae subfamily. For example, in some embodiments, the viral respiratory illness may be caused by a severe acute respiratory syndrome-related coronavirus (SARS), which may be SARS-CoV or SARS-CoV-2.

In some embodiments, administering a thiazolide agent, such as nitazoxanide and/or tizoxanide and/or its salt, may result in a statistically significant reduction of probability for the subject to be affected by the viral respiratory illness. In other words, a proportion of subjects affected by the viral respiratory illness will be statistically significantly lower, such as by at least 20% or at least 30% or at least 40% or at least 50% or at least 60% or at least 70% or at least 80%, for a population for which the thiazolide agent was administered compared to a population, which did not receive the thiazolide agent, such as a population receiving a placebo. For example, for a viral respiratory illness caused by a virus belonging to the Coronaviridae family, such as SARS-CoV or SARS-CoV-2, a proportion of subjects, who display one or more symptoms of the viral respiratory illness and for whom the virus belonging to the Coronaviridae family, such as SARS-CoV or SARS-CoV-2, as a cause of the illness is confirmed, for example, through a nasopharyngeal swab, a saliva test or another laboratory test, will be statistically significantly lower such as by at least 20% or at least 30% or at least 40% or at least 50% or at least 60% or at least 70% or at least 80%, than for a population for which the thiazolide agent was administered compared to a population, which did not receive the thiazolide agent, such as a population receiving a placebo.

In some embodiments, for a viral respiratory infection caused by a virus belonging to the Coronaviridae family, such as SARS-CoV or SARS-CoV-2, a proportion of subjects, who are hospitalized due to the viral respiratory infection and for whom the virus belonging to the Coronaviridae family, such as SARS-CoV or SARS-CoV-2, as a cause of the infection is confirmed, for example, through a nasopharyngeal swab, a saliva test or another laboratory test, will be statistically significantly lower such as by at least 20% or at least 30% or at least 40% or at least 50% or at least 60% or at least 70% or at least 80%, than for a population for which the thiazolide agent was administered compared to a population, which did not receive the thiazolide agent, such as a population receiving a placebo.

In some embodiments, for a viral respiratory infection caused by a virus belonging to the Coronaviridae family, such as SARS-CoV or SARS-CoV-2, a proportion of subjects, who will die due to the viral respiratory infection and for whom the virus belonging to the Coronaviridae family, such as SARS-CoV or SARS-CoV-2, as a cause of the infection is confirmed, for example, through a nasopharyngeal swab, a saliva test or another laboratory test, will be statistically significantly lower such as by at least 20% or at least 30% or at least 40% or at least 50% or at least 60% or at least 70% or at least 80%, than for a population for which the thiazolide agent was administered compared to a population, which did not receive the thiazolide agent, such as a population receiving a placebo.

In some embodiments, for a viral respiratory infection caused by a virus belonging to the Coronaviridae family, such as SARS-CoV or SARS-CoV-2, a proportion of subjects, who test positive for antibodies to the virus belonging to the Coronaviridae family, such as SARS-CoV or SARS-CoV-2, will be statistically significantly lower such as by at least 20% or at least 30% or at least 40% or at least 50% or at least 60% or at least 70% or at least 80%, than for a population for which the thiazolide agent was administered compared to a population, which did not receive the thiazolide agent, such as a population receiving a placebo.

Administration of the thiazolide agent for prevention purposes may use the compositions, doses and administration regiments described above.

In some embodiments, administration of the thiazolide agent for prevention purposes may be for a period of at least five days, or at least 7 days or at least 10 days or at least 14 days or at least 21 days or at least 28 days or at least 35 days or at least 42 days or at least 49 days or least 56 days.

Embodiments described herein are further illustrated by, though in no way limited to, the following working examples.

### Example 1

### Introduction

A randomized double-blind clinical trial was conducted to evaluate the safety and effectiveness of nitazoxanide 300 mg extended release tablets administered 600 mg orally twice daily compared to a placebo in treating subjects with influenza and/or influenza-like illness.

### Materials and Methods

Subjects at least 12 years of age with influenza-like illness characterized by oral temperature ≥99.4° F., at least one respiratory symptom (cough, sore throat or nasal congestion) and one constitutional symptom (fatigue, headache, myalgia or feverishness) were enrolled within 40 hours of symptom onset at 57 outpatient clinics in the United States, Puerto Rico and Australia when influenza had been confirmed in the community. Subjects expected to require hospital care, subjects with moderate or severe asthma, cystic fibrosis, COPD, congestive heart failure, cardiac arrhythmia or other conditions placing them at risk of influenza complications, females who were pregnant, breastfeeding or of child-bearing potential without adequate birth control, and subjects receiving influenza antivirals within three days prior to screening were excluded. Subjects returned to the clinic on study days 7 and 22 for follow-up including physical examination, collection of nasopharyngeal swabs (one from each nostril), collection of blood and urine samples for laboratory safety tests, and review of compliance, concomitant medications and adverse events. Baseline and follow-up nasopharyngeal swabs were subjected to RT-PCR testing using the ePlex® Respiratory Pathogen Panel (Genmark, Carlsbad, California) to detect influenza A (non-specific as to subtype), influenza A/H1, A/H1N1 (2009), A/H3 subtypes, influenza B, respiratory syncytial virus A and B (RSV), parainfluenza 1, 2, 3 and 4, human metapneumovirus (hMPV), adenovirus (A-F), human rhinovirus/enterovirus, coronaviruses NL63, HKU1, 229E and OC43, Chlamydophila pneumoniae, and Mycoplasma pneumoniae. Subjects completed a FLU-PRO© diary¹⁻³ at the baseline visit and then every evening between 7 and 11 pm through study day 21. At the same time, they also completed an Activity Assessment using an 11-point visual analog scale (0=unable to perform normal activity, 10=fully able to perform normal activity)⁴ and recorded any adverse experiences. Diary information was entered into an electronic application downloaded to the subjects' smart phone or an electronic device provided by the study site. Time from first dose until the subject reported usual health and ability to perform all normal activities for two consecutive daily diary entries were analyzed using Kaplan-Meier survival curves and a Prentice-Wilcoxon test. Other analyses of time to Sustained Response were also performed using Kaplan-Meier survival curves and a Prentice-Wilcoxon test. For this purpose, Sustained Response was defined as: (i) reduction of total FLU-PRO symptom score from the prior day, (ii) subject reports overall symptoms are either “somewhat better” or “much better” than yesterday, (iii) no oral temperature ≥100.4° C. during last 24 hours, (iv) no subsequent increase in any mean domain or subdomain score during the 21-day study period except under background levels where such background levels are body/systemic ≤0.56, throat ≤0.67, eyes ≤0.67, gastrointestinal ≤2.0, head ≤2.0, nose ≤0.75, chest=0, cough ≤1.75).

### Results

1,032 subjects were enrolled in the clinical trial. Baseline nasopharyngeal swabs from 92 subjects tested positive for coronavirus infection by RT-PCR, 65 of whom tested positive for coronavirus as the sole pathogen. Demographic and disease-related characteristics of this population are presented in Table 1.

TABLE 1
Demographic and Disease-Related Characteristics of Subjects
with Laboratory-confirmed Coronavirus Infection at Baseline

| | | | | | All | |
|---|---|---|---|---|---|---|
| All Coronavirus-infected | NTZ | | Placebo | | Subjects | |
| (including co-infections) | (N = 51) | | (N = 41) | | (N = 92) | |
| Male, N (%) | 22 | (43%) | 13 | (32%) | 35 | (38%) |
| Age (years), mean (SD) | 34.0 | (13.97) | 39.3 | (14.92) | 36.3 | (14.56) |
| Weight (kg), mean (SD) | 87.7 | (30.57) | 87.1 | (24.92) | 87.5 | (28.04) |
| BMI (kg/m²), mean (SD) | 31.4 | (9.97) | 30.4 | (8.27) | 30.9 | (9.22) |
| Race, N Black/Hispanic/White/Other | 5/17/24/5 | | 4/11/26/0 | | 9/28/50/5 | |
| Past or Present Smoker, N (%) | 11 | (21%) | 14 | (34%) | 25 | (27%) |
| In-Office Temperature (C.), mean (SD) | 37.9 | (0.41) | 37.9 | (0.59) | 37.9 | (0.50) |
| Co-infections: | | | | | | |
| Influenza A | 11 | (22%) | 6 | (15%) | 17 | (18%) |
| Enterovirus/Rhinovirus | 2 | (4%) | 5 | (12%) | 7 | (8%) |
| Other | 3 | (6%)¹ | 2 | (5%)² | 5 | (5%) |

| Subjects with Coronavirus as Sole Pathogen | (N = 35) | | (N = 28) | | (N = 63) | |
|---|---|---|---|---|---|---|
| Male, N (%) | 12 | (34%) | 10 | (36%) | 22 | (35%) |
| Age (years), mean (SD) | 33.2 | (12.36) | 42.4 | (15.23) | 37.3 | (14.34) |
| Weight (kg), mean (SD) | 88.8 | (33.35) | 88.5 | (28.16) | 88.7 | (30.9) |
| BMI (kg/m²), mean (SD) | 31.6 | (11.15) | 31.2 | (9.51) | 31.4 | (10.37) |
| Race, N Black/Hispanic/White/Other | 5/10/16/4 | | 4/8/16/0 | | 9/18/32/4 | |
| Past or Present Smoker, N (%) | 7 | (20%) | 12 | (43%) | 19 | (30%) |
| In-Office Temperature (C), mean (SD) | 37.8 | (0.34) | 37.9 | (0.63) | 37.9 | (0.49) |

¹One with adenovirus, one RSV B, one parainfluenza 3
²One with influenza B, one Chlamydophila pneumoniae

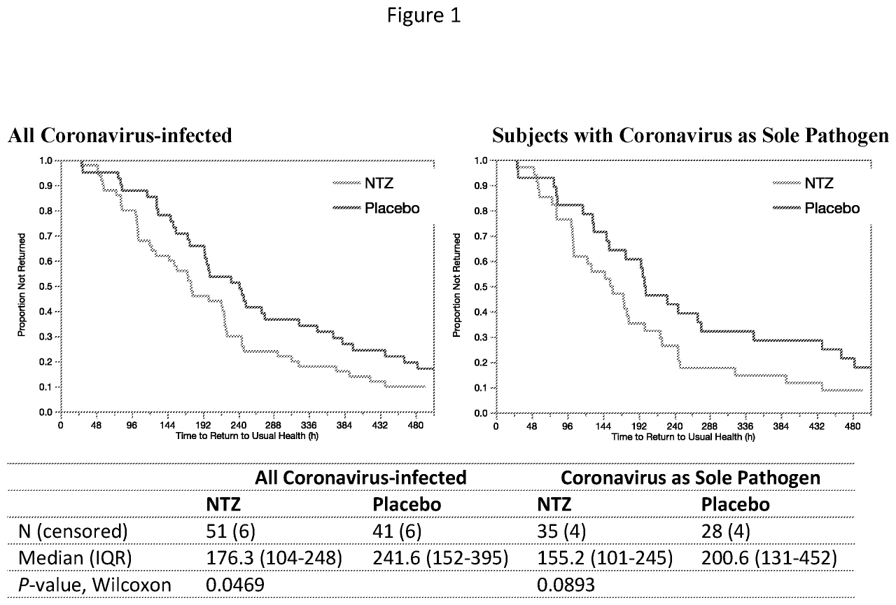

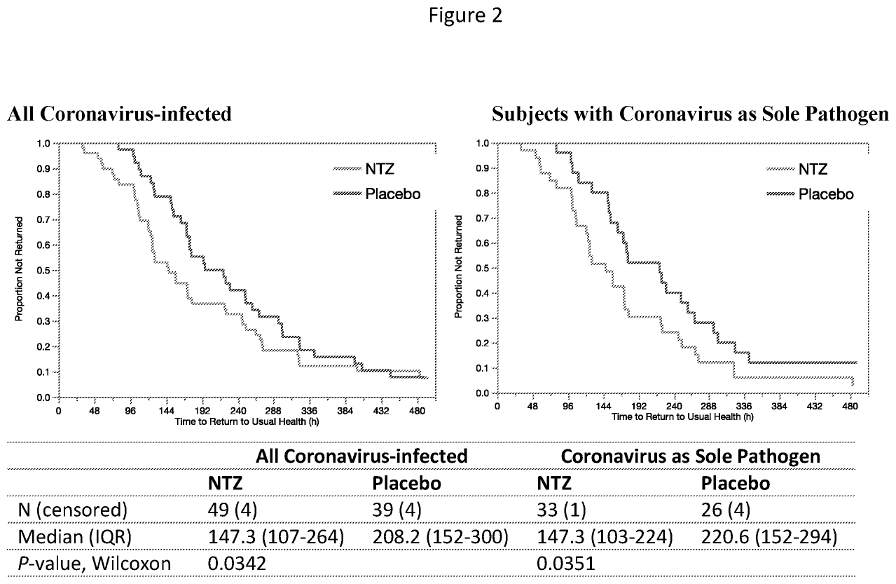

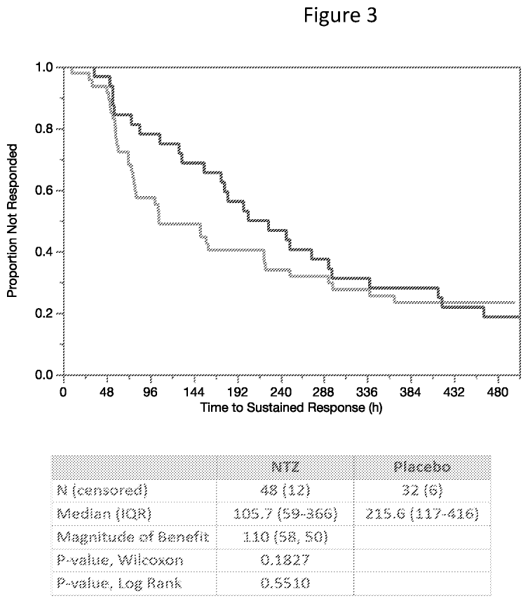

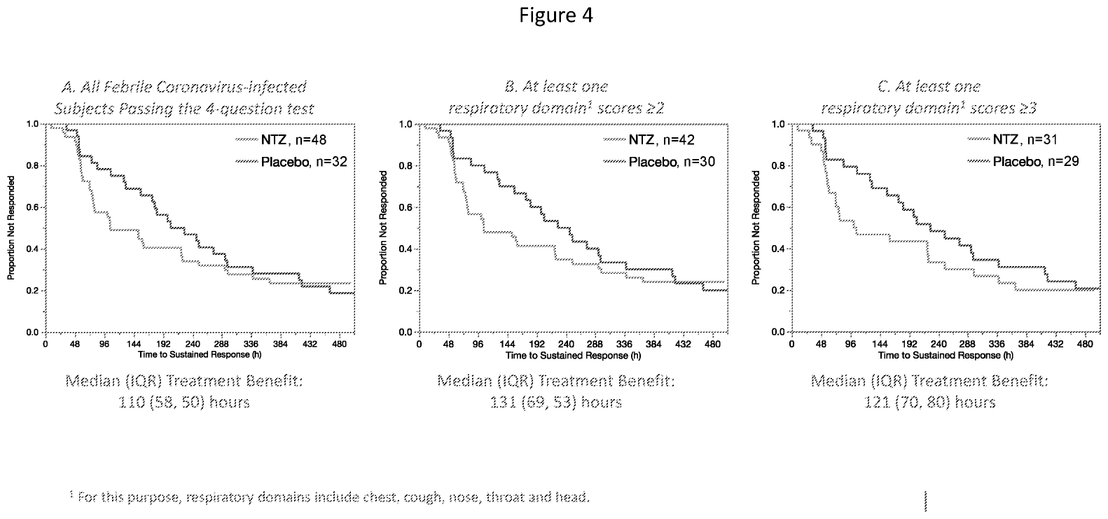

Nitazoxanide-treated subjects with laboratory-confirmed coronavirus infection at baseline reported reductions of time from first dose to return to usual health and time from first dose to return to ability to perform all normal activities. See FIGS. 1 and 2. Likewise, nitazoxanide-treated subjects with laboratory-confirmed coronavirus infection at baseline reported reductions of time from first dose to Symptom Response with greater magnitudes of treatment observed for subjects with more severe respiratory symptoms. See FIGS. 3 and 4.

Nitazoxanide was well tolerated by the patients. Adverse events reported by at least 2% of subjects were mild chromaturia (14.6% vs. 1.0% for placebo) and diarrhea (6.6% vs. 4.9% for placebo).

### Conclusion

In this clinical trial, treatment of febrile subjects with laboratory-confirmed coronavirus infection with nitazoxanide 300 mg extended release tablets administered 600 mg orally twice daily for five days was associated with improvement in time to return to usual health and time until subjects are able to perform normal activities. The treatment regimen was well-tolerated by patients.

### REFERENCES

1. Powers J H, et al. BMC Infect Dis 2015; 16:1.
2. Powers J H, et al. Value Health 2017; 21:210-18.
3. Powers J H, et al. PLoS One 2018; 13:e0194180.
4. Osborne R H, et al. J Outcomes Res 2000; 4:15-30.

### Example 2

### A Randomized, Double-Blind, Placebo Controlled Trial to Evaluate the Efficacy and Safety of Nitazoxanide (NTZ) for Post-Exposure Prophylaxis of COVID-19 and Other Viral Respiratory Illnesses in Elderly Residents of Long-Term Care Facilities (LTCF)

| | | |
|---|---|---|
| Indication: | | Post-exposure prophylaxis of COVID-19 and other |
| | | VRIs |
| Design: | | Multicenter, randomized, double-blind, placebo- |
| | | controlled trial to evaluate the efficacy and safety of |
| | | NTZ for post-exposure prophylaxis of COVID-19 and |
| | | other VRIs in elderly LTCF residents. |
| Population: | | Males and females ≥65 years of age residing in LTCFs |
| Randomization: | | 1:1 within stratum (LTCF) at the subject level |
| Study Dose and Administration: | | Group 1 (NTZ): Two NTZ 300 mg tablets orally twice |
| | | daily (b.i.d.) for 6 weeks. |
| | | Group 2 (Placebo): Two placebo tablets orally b.i.d. for 6 weeks. |
| | | All subjects will receive a B complex vitamin (Super B- |
| | | Complex ™, Igennus Healthcare Nutrition, Cambridge, |
| | | UK) one tablet twice daily to mask potential |
| | | chromaturia that may be associated with NTZ. |
| Objective: | | Evaluate the effect of NTZ administered 600 mg orally |
| | | b.i.d. for 6 weeks in preventing symptomatic laboratory- |
| | | confirmed COVID-19 and other VRIs compared to that |
| | | of a placebo. |
| Primary Efficacy Parameters: | i. | The proportion of subjects with symptomatic |
| | | laboratory-confirmed COVID-19 identified after |
| | | start of treatment and before the end of the 6-week |
| | | treatment period. |
| | ii. | The proportion of subjects with symptomatic |
| | | laboratory-confirmed VRI identified after the start |
| | | of treatment and before the end of the 6-week |
| | | treatment period |
| Secondary Efficacy Parameters: | i. | The proportion of subjects hospitalized due to |
| | | COVID-19 or complications thereof. |
| | ii. | Mortality due to COVID-19 or complications |
| | | thereof. |
| | iii. | The proportion of subjects (with or without |
| | | symptoms) testing positive for antibodies to SARS- |
| | | CoV-2 at either of the Week 6 or Week 8 visits. |

| | |
|---|---|
| Exploratory Efficacy Parameters: | Proportion of subjects hospitalized due to viral |
| | respiratory illness (VRIs) or complications thereof; |
| | mortality due to VRIs or complications thereof; |
| | proportion of subjects experiencing acute respiratory |
| | illness (ARI); proportion of subjects hospitalized due to |
| | ARI or complications thereof, and mortality due to ARI |
| | or complications thereof. |

### Study Objectives

The primary objectives of this study are to evaluate the effect of NTZ administered orally 600 mg twice daily for 6 weeks in preventing symptomatic laboratory-confirmed COVID-19 and other VRIs in elderly LTCF residents compared to that of a placebo.

Secondary objectives are to evaluate the effect on (i) hospitalization due to COVID-19 or complications thereof, (ii) mortality due to COVID-19 or complications thereof, and (iii) the proportion of subjects (with or without symptoms) testing positive for antibodies to SARS-CoV-2 at either of the Week 6 or Week 8 visits.

Exploratory objectives include (i) hospitalization due to VRI or complications thereof, (ii) mortality due to VRI or complications thereof, (iii) proportions of subjects experiencing acute respiratory illness (ARI), (iv) hospitalization due to ARI or complications thereof, and (v) mortality due to ARI or complications thereof.

Other important objectives include evaluation of the safety of NTZ by analysis of adverse events and evaluation of relationships between pharmacokinetics and clinical or virologic responses.

### Study Design

The study will be a multicenter, randomized, double-blind, placebo-controlled trial to evaluate the efficacy and safety of NTZ for post-exposure prophylaxis of COVID-19 and other VRIs in elderly LTCF residents.

Subjects will be randomized within strata 1:1 to one of the following groups:
Group 1 (NTZ): Two NTZ 300 mg tablets b.i.d. for 6 weeks
Group 2 (Placebo): Two placebo tablets b.i.d. for 6 weeks

Choice of Patient-Reported Outcome Instrument. This clinical trial will use the InFLUenza Patient-Reported Outcome Questionnaire (FLU-PRO©). FLU-PRO© was developed with the support of the U.S. Department of Health and Human Services through the National Cancer Institute and the National Institutes of Allergy and Infectious Diseases, National Institutes of Health, in response to the need for improved metrics to evaluate treatment effect in clinical trials of drugs for the treatment of influenza and other respiratory tract viral diseases. It was developed and validated in accordance with FDA's guidance, “Patient-Reported Outcome Measures: Use in Medical Product Development to Support Labeling Claims.” FLU-PRO© was separately validated for use in a population with non-influenza like illness and also has recently been used in vaccine studies with older adults for the prevention of RSV (Powers et al. 2018, Yu et al. 2020).

Choice of ARI Definition. The primary endpoint of this trial may require a determination that an ARI has occurred. ARI is defined as “≥0.5 increase from baseline in mean symptom score for the chest/respiratory FLU-PRO domain or ≥0.5 increase from baseline in mean symptom score for at least two of the following FLU-PRO© domains: body/systemic, nose, throat.” The ARI definition may require an increase in symptom scores from baseline, although the magnitude of the increase (≥0.5) in mean domain scores is low, and it may be required that the increase in mean score be achieved for only one or two of the four domains.

TABLE 2
Clinical Symptoms Required for Suspected ARI1 (adapted from Yu et al. 2020)

| Upper Respiratory | Lower Respiratory | Systemic |
|---|---|---|
| Symptoms | Symptoms | Symptoms |
| Nasal | Cough (coughing, chest | Myalgias or arthralgias |
| congestion/rhinorrhea | congestion, chest tightness, | (body aches or pains) ² |
| (runny or dripping nose, | dry or hacking cough, wet | Fatigue (weak or tired, |
| congested or stuffy nose, | or loose cough) ² | sleeping more than usual) ² |
| head congestion, sinus | Dyspnea (shortness of | Headache |
| pressure) ² | breath) ² | Decreased appetite (lack of |
| Sore throat (sore or | Sputum (coughing up | appetite, did not feel like |
| painful throat) ² | sputum or phlegm) ² | eating) ² |
| | Wheezing | Feverishness (felt hot, |
| | | chills or shivering, felt |
| | | cold, sweating) ² |

¹ Suspected ARI requires self-reporting of any Lower Respiratory Symptom, or at least one Upper Respiratory Symptom together with one Systemic Symptom.
² Lay language used in the FLU-PRO © questionnaire is presented in parentheses.

Recent reports of COVID-19 in long-term care facilities in the United States have suggested a high rate of transmission of the SARS-CoV-2 virus, but infection control procedures may significantly decrease transmission (McMichael et al. 2020, Kimball et al. 2020). A recent study of 264 elderly volunteers (≥65 years of age) reported 1.6 respiratory tract infections (not laboratory-confirmed) per person-year indicating approximately a 20% probability of respiratory tract infection over a given 6-week period (Mannick et. al, 2018). Infection control procedures in place during the period covered by this clinical trial are also expected to reduce the transmission of other respiratory viruses.

Effective prophylaxis may reduce the rate of COVID-19 and other VRIs by at least 40% or by at least 50% or by at least 60% or by at least 70% or by at least 80%.

### Efficacy Variables

Primary Efficacy Parameters: i. The proportion of subjects with symptomatic laboratory-confirmed COVID-19 identified after start of treatment and before the end of the 6-week treatment period; ii. The proportion of subjects with symptomatic laboratory-confirmed VRI identified after start of treatment and before the end of the 6-week treatment period
Secondary Efficacy Parameters: i. The proportion of subjects hospitalized due to COVID-19 or complications thereof; ii. Mortality due to COVID-19 or complications thereof; iii. The proportion of subjects (with or without symptoms) testing positive for antibodies to SARS-CoV-2 at either of the Week 6 or Week 8 visits.
Exploratory Efficacy Parameters: Hospitalization due to VRI or complications thereof, mortality due to VRI or complications thereof, proportions of subjects experiencing acute respiratory illness (ARI), hospitalization due to ARI or complications thereof, and mortality due to ARI or complications thereof.

### Response Definitions

ARI: ≥0.5 increase from baseline in mean symptom score for the chest/respiratory FLU-PRO domain or ≥0.5 increase from baseline in mean symptom score for at least two of the following FLU-PRO© domains: body/systemic, nose, throat.
COVID-19: ARI after start of treatment and before the end of the 6-week treatment period associated with detection of SARS-CoV-2 by RT-PCR assay of nasopharyngeal swab.
VRI: ARI after start of treatment and before the end of the 6-week treatment period associated with detection of any respiratory virus by RT-PCR assay of nasopharyngeal swab.

### Exploratory Analyses Will be Performed as Follows

Proportions of subjects experiencing each of the following will be compared by treatment group using a two-sided CMH Chi-square test (unadjusted α=0.05): (i) hospitalization due to VRI or complications thereof; (ii) mortality due to VRI or complications thereof, (iii) acute respiratory illness (ARI), (vi) hospitalization due to ARI or complications thereof; and (v) mortality due to ARI or complications thereof.

### Efficacy Analyses

Efficacy analyses will be based on a population consisting of all subjects randomized without a laboratory-detected viral respiratory infection at the baseline visit (intention-to-treat or ITT population). All chi-square analyses will be calculated with appropriate continuity corrections.

There will be two primary efficacy analyses:

The proportion of subjects experiencing COVID-19 in the NTZ treatment group will be compared to that of the placebo treatment group using a two-sided Cochran-Mantel-Haenszel (CMH) chi-square test (α=0.049).

The proportion of subjects experiencing VRI in the NTZ treatment group will be compared to that of the placebo treatment group using a two-sided CMH chi-square test (α=0.001).

Secondary Analyses Will be Performed as Follows:

The proportion of subjects experiencing COVID-19 or VRI for treatment vs control will be tested within strata using Pearson chi-square tests at an unadjusted □=0.05.

Confidence intervals (95% confidence level) will be produced for the overall odds ratio and for the odds ratios for NTZ vs placebo for each stratum.

Proportions of subjects experiencing mortality due to COVID-19 or complications thereof will be compared by treatment group using a two-sided Pearson chi-square test (unadjusted α=0.05).

Proportions of subjects (with or without symptoms) testing positive for antibodies to SARS-CoV-2 at either of the Week 6 or Week 8 visits will be compared by treatment group using a two-sided Pearson chi-square test (unadjusted α=0.05).

Exploratory Analyses Will be Performed as Follows:

Proportions of subjects experiencing each of the following will be compared by treatment group using a two-sided Pearson chi-square test (unadjusted α=0.05): (i) hospitalization due to VRI or complications thereof, (ii) mortality due to VRI or complications thereof, (iii) proportions of subjects experiencing acute respiratory illness (ARI), (vi) hospitalization due to ARI or complications thereof, (v) mortality due to ARI or complications thereof.

### Drug Regimens, Administration and Duration

Group 1 (NTZ): Subjects will receive two NTZ 300 mg tablets b.i.d. with food (<1 hour after food intake) and a B complex vitamin (Super B-Complex™, Igennus Healthcare Nutrition, Cambridge, UK) b.i.d. for 6 weeks.
Group 2 (Placebo): Subjects will receive two placebo tablets b.i.d. with food (<1 hour after food intake) and a B complex vitamin (Super B-Complex™, Igennus Healthcare Nutrition, Cambridge, UK) b.i.d. for 6 weeks.

The food prior to drug intake should preferably be a high-fat meal, but at minimum a cereal bar.

All subjects will receive a vitamin B complex supplement one tablet twice a day (manufacturer's labeled dosing) to help mask any potential chromaturia attributed to NTZ and aid in maintaining study blinding.

### Example 3

### A Randomized, Double-Blind, Placebo Controlled Trial to Evaluate the Efficacy and Safety of Nitazoxanide (NTZ) for Post-Exposure Prophylaxis of COVID-19 and Other Viral Respiratory Illnesses (VRI) in Healthcare Workers

| | | |
|---|---|---|
| Indication: | | Post-exposure prophylaxis of COVID-19 and other |
| | | VRIs |
| Design: | | Multicenter, randomized, double-blind, placebo- |
| | | controlled trial to evaluate the efficacy and safety of |
| | | NTZ for post-exposure prophylaxis of COVID-19 and |
| | | other VRIs. |
| Population: | | Healthcare workers at increased risk of occupational |
| | | exposure to COVID-19 |
| Randomization: | | 1:1 at the subject level |
| Study Dose and Administration: | | Group 1 (NTZ): Two NTZ 300 mg tablets orally twice |
| | | daily (b.i.d.) for 6 weeks. |
| | | Group 2 (Placebo): Two placebo tablets orally b.i.d. |
| | | for 6 weeks. |
| | | All subjects will receive a B complex vitamin (Super |
| | | B-Complex ™, Igennus Healthcare Nutrition, |
| | | Cambridge, UK) one tablet twice daily to mask |
| | | potential chromaturia that may be associated with |
| | | NTZ. |
| Objective: | | Evaluate the effect of NTZ administered 600 mg orally |
| | | b.i.d. for 6 weeks in preventing symptomatic |
| | | laboratory-confirmed COVID-19 and other VRIs |
| | | compared to that of a placebo. |
| Primary Efficacy Parameters: | i. | The proportion of subjects with symptomatic |
| | | laboratory-confirmed COVID-19 identified after |
| | | start of treatment and before the end of the 6-week |
| | | treatment period. |
| | ii. | The proportion of subjects with symptomatic |
| | | laboratory-confirmed VRI identified after the start |
| | | of treatment and before the end of the 6-week |
| | | treatment period |
| Secondary Efficacy Parameters: | i. | Mortality due to COVID-19 or complications |
| | | thereof. |
| | ii. | The proportion of subjects (with or without |
| | | symptoms) testing positive for antibodies to SARS- |
| | | CoV-2 at either of the Week 6 or Week 8 visits. |

| | |
|---|---|
| Exploratory Efficacy Parameters: | Proportion of subjects hospitalized due to viral |
| | respiratory illness (VRI) or complications thereof, |
| | mortality due to VRI or complications thereof; |
| | proportion of subjects experiencing acute respiratory |
| | illness (ARI); proportion of subjects hospitalized due |
| | to ARI or complications thereof, mortality due to ARI |
| | or complications thereof. |

### Study Objectives

The primary objectives of this study are to evaluate the effect of NTZ administered orally 600 mg twice daily for 6 weeks in preventing symptomatic laboratory-confirmed COVID-19 and other VRIs in healthcare workers at high risk of occupational exposure compared to that of a placebo.

The secondary objectives of this study are to evaluate the effect of NTZ administered orally 600 mg twice daily for 6 weeks in (i) preventing mortality due to COVID-19 or complications thereof, and (ii) reducing the proportion of subjects (with or without symptoms) testing positive for antibodies to SARS-CoV-2 at either of the Week 6 or Week 8 visits.

Exploratory objectives include (i) hospitalization due to VRI or complications thereof, (ii) mortality due to VRI or complications thereof, (iii) proportions of subjects experiencing acute respiratory illness (ARI), (iv) hospitalization due to ARI or complications thereof, and (v) mortality due to ARI or complications thereof.

Other important objectives include evaluation of the safety of NTZ by analysis of adverse events and evaluation of relationships between pharmacokinetics and clinical or virologic responses.

### Study Design

The study will be a multicenter, stratified, randomized, double-blind, placebo-controlled trial to evaluate the efficacy and safety of NTZ for post-exposure prophylaxis of COVID-19 and other VRIs.

Subjects will be stratified according to their workspace, which is a proxy for exposure to SARS-CoV-2 and other VRIs. The strata are:
Emergency Department
Intensive Care Unit
COVID-specific Care Unit
Walk-in Clinic
Paramedics and other first responders (e.g., firefighter/EMT)

Within strata, subjects will be randomized 1:1 to one of the following groups:
Group 1 (NTZ): Two NTZ 300 mg tablets b.i.d. for 6 weeks
Group 2 (Placebo): Two placebo tablets b.i.d. for 6 weeks

Choice of Patient-Reported Outcome Instrument. This clinical trial will use the InFLUenza Patient-Reported Outcome Questionnaire)(FLU-PRO©) . FLU-PRO© was developed with the support of the U.S. Department of Health and Human Services through the National Cancer Institute and the National Institutes of Allergy and Infectious Diseases, National Institutes of Health, in response to the need for improved metrics to evaluate treatment effect in clinical trials of drugs for the treatment of influenza and other respiratory tract viral diseases. It was developed and validated in accordance with FDA's guidance, “Patient-Reported Outcome Measures: Use in Medical Product Development to Support Labeling Claims.” It has also been validated for use on an electronic device as part of an electronic diary (“eDiary”) that can time stamp diary entries to ensure timely recording, thereby mitigating risks of recall bias. We will use an electronic diary for this clinical trial. FLU-PRO© was separately validated for use in a population with non-influenza like illness and also has recently been used in vaccine studies with older adults for the prevention of RSV (Powers et al. 2018, Yu et al. 2020).

Choice of ARI Definition. The primary endpoint of this trial requires a determination that an ARI has occurred. We have defined ARI as “≥0.5 increase from baseline in mean symptom score for the chest/respiratory FLU-PRO domain or ≥0.5 increase from baseline in mean symptom score for at least two of the following FLUPRO© domains: body/systemic, nose, throat.” In arriving at this definition, we reviewed published mean FLU-PRO© symptom score data over the course of RSV illness (Yu et al. 2020) as well as data from recently completed clinical trials in approximately 3,000 subjects with colds and influenza. The ARI definition may require an increase in symptom scores from baseline, although the magnitude of the required increase (≥0.5) in mean domain scores is low, and it may be required that the increase in mean score be achieved for only one or two of the four domains.

TABLE 3
Clinical Symptoms Required for Suspected ARI1 (adapted from Yu et al. 2020)

| Upper Respiratory | Lower Respiratory | Systemic |
|---|---|---|
| Symptoms | Symptoms | Symptoms |
| Nasal | Cough (coughing, chest | Myalgias or arthralgias |
| congestion/rhinorrhea | congestion, chest tightness, | (body aches or pains) ² |
| (runny or dripping nose, | dry or hacking cough, wet | Fatigue (weak or tired, |
| congested or stuffy nose, | or loose cough) ² | sleeping more than usual) ² |
| head congestion, sinus | Dyspnea (shortness of | Headache |
| pressure) ² | breath) ² | Decreased appetite (lack of |
| Sore throat (sore or | Sputum (coughing up | appetite, did not feel like |
| painful throat) ² | sputum or phlegm) ² | eating) ² |
| | Wheezing | Feverishness (felt hot, |
| | | chills or shivering, felt |
| | | cold, sweating) ² |

¹ Suspected ARI requires self-reporting of any Lower Respiratory Symptom, or at least one Upper Respiratory Symptom together with one Systemic Symptom.
² Lay language used in the FLU-PRO © questionnaire is presented in parentheses.

The effective prophylaxis may result in at least 40%, or at least 50% or at least 60% or at least 70% or at least 80% reduction of the illness rate.

### Efficacy Variables

Primary Efficacy Parameters: i. The proportion of subjects with symptomatic laboratory-confirmed COVID-19 identified after start of treatment and before the end of the 6-week treatment period
ii. The proportion of subjects with symptomatic laboratory-confirmed VRI identified after start of treatment and before the end of the 6-week treatment period

Secondary Efficacy Parameters: i. Mortality due to COVID-19 or complications thereof
ii. The proportion of subjects (with or without symptoms) testing positive for antibodies to SARS-CoV-2 at either of the Week 6 or Week 8 visits

Exploratory Efficacy Parameters: Hospitalization due to VRI or complications thereof, mortality due to VRI or complications thereof, proportions of subjects experiencing acute respiratory illness (ARI), hospitalization due to ARI or complications thereof, mortality due to ARI or complications thereof.

### Response Definitions

ARI: ≥0.5 increase from baseline in mean symptom score for the chest/respiratory FLU-PRO domain or ≥0.5 increase from baseline in mean symptom score for at least two of the following FLU-PRO© domains: body/systemic, nose, throat.
COVID-19: ARI after start of treatment and before the end of the 6-week treatment period associated with detection of SARS-CoV-2 by RT-PCR assay of nasopharyngeal swab.
VRI: ARI after start of treatment and before the end of the 6-week treatment period associated with detection of any respiratory virus by RT-PCR assay of nasopharyngeal swab.

### Efficacy Analyses

Efficacy analyses will be based on a population consisting of all subjects randomized without a laboratory-detected viral respiratory infection at the baseline visit (intention-to-treat or ITT population). All chi-square analyses will be calculated with appropriate continuity corrections.

There will be two primary efficacy analyses:

The proportion of subjects experiencing COVID-19 in the NTZ treatment group will be compared to that of the placebo treatment group using a two-sided Cochran-Mantel-Haenszel (CMH) chi-square test (α=0.049).

The proportion of subjects experiencing VRI in the NTZ treatment group will be compared to that of the placebo treatment group using a two-sided CMH chi-square test (α=0.001).

Secondary Analyses Will be Performed as Follows:

The proportion of subjects experiencing COVID-19 or VRI for treatment vs control will be tested within strata using Pearson chi-square tests at an unadjusted □=0.05.

Confidence intervals (95% confidence level) will be produced for the overall odds ratio and for the odds ratios for NTZ vs placebo for each stratum.

Proportions of subjects experiencing mortality due to COVID-19 or complications thereof will be compared by treatment group using a two-sided Pearson chi-square test (unadjusted α=0.05).

Proportions of subjects (with or without symptoms) testing positive for antibodies to SARS-CoV-2 at either of the Week 6 or Week 8 visits will be compared by treatment group using a two-sided Pearson chi-square test (unadjusted α=0.05).

Exploratory analyses will be performed as follows:

Proportions of subjects experiencing each of the following will be compared by treatment group using a two-sided Pearson chi-square test (unadjusted α=0.05): (i) hospitalization due to VRI or complications thereof, (ii) mortality due to VRI or complications thereof, (iii) proportions of subjects experiencing acute respiratory illness (ARI), (vi) hospitalization due to ARI or complications thereof, (v) mortality due to ARI or complications thereof.

### Drug Regimens, Administration and Duration

Group 1 (NTZ): Subjects will receive two NTZ 300 mg tablets b.i.d. with food (<1 hour after food intake) and a B complex vitamin (Super B-Complex™, Igennus Healthcare Nutrition, Cambridge, UK) b.i.d. for 6 weeks.
Group 2 (Placebo): Subjects will receive two placebo tablets b.i.d. with food (<1 hour after food intake) and a B complex vitamin (Super B-Complex™, Igennus Healthcare Nutrition, Cambridge, UK) b.i.d. for 6 weeks.

The food prior to drug intake should preferably be a high-fat meal, but at minimum a cereal bar.

All subjects will receive a vitamin B complex supplement one tablet twice a day (manufacturer's labeled dosing) to help mask any potential chromaturia attributed to NTZ and aid in maintaining study blinding.

### REFERENCES FOR EXAMPLES 2 AND 3

1 Anderson V R, Curran M P. Drugs 2007; 67:1947-67.
2. ALINIA (nitazoxanide) prescribing information. Romark, L. C., Tampa, FL, June 2019.
3. Ashton L V, et al. Vet Med Int 2010; 2010:891010.
4. Backman J T, et al. Pharmacol Rev 2016; 68:168-241.
5. Braakman I, et al. Nature 1992; 356:260-62.
6. Braakman I, et al. J Cell Biol 1991; 114:401-11.
7. Cao J, et al. Antiviral Res 2015; 114:1-10.
8. Centers for Disease Control and Prevention. Atlanta, GA. Interim Guidance for Influenza Outbreak Management in Long-Term Care and Post-Acute Care Facilities [Internet]. [updated 18 Nov. 2019; cited 12 Mar. 2020]. Available from: https://www.cdc.gov/flu/professionals/infectioncontrol/ltc-facility-guidance.htm
9. Chang C W, et al. J Biomed Sci 2009; 16:80.
10. Checovich M M, et al. J Am Med Dir Assoc 2020; 21:29-33.
11. Childs A, et al. BMC Geriatrics 2019; 19:210.
12. Doms R W, et al. J Cell Biol 1987; 105:1957-69.
13. Falsey A R, et al. J Am Geriatr Soc 2008; 56:1281-85.
14. Fendrick A M, et al. Arch Intern Med 2003; 163:487-94.
15. Goodwin K, et al. Vaccine 2006; 24:1159-69.
16. Hand J, et al. Emerg Infect Dis 2018; 24:1964-66.
17. Hayden F G, et al. N Engl J Med 1999; 341:1336-43.
18. Hong S K, et al. Int Immunopharmacol 2012; 13:23-27.
19. Jain S, Self W H, et al. N Engl J Med 2015; 373:415-27.
20. Kimball A, et al MMWR Morb Mortal Wkly Rep 2020; 69:377-381.
21. Kirkpatrick G L. Prim Care 1996; 23:657-75.
22. Lee J H, et al. Int J Obes 2017; 41:645-51.
23. Longtin J, et al. Emerg Infect Dis 2010; 16:1463-65.
24. Mannick J B, et al. Sci Transl Med 2018; 10:eaaq1564.
25. McMichael T M, et al. MMWR Morb Mortal Wkly Rep 2020; 69:339-342.
26. Mirazimi A, Svensson L. J Virol 2000; 74:8048-52.
27. McGeer A, et al. Can J Infect Dis 2000; 11:187-92.
28. Monto A S, et al. J Infect Dis 1987; 156:43-49.
29. Peters P H Jr, et al. J Am Geriatr Soc 2001; 49:1025-31.
30. Piacentini S, et al. Sci Rep 2018; 8:10425.
31. Powers J H, et al. PLoS ONE 2018 13(3):d0194180.
32. RELENZA (zanamivir) prescribing information. GlaxoSmithKline, Research Triangle Park, NC, June 2018.
33. Rossignol J F, van Baalen C. [Abstract]. Presented at the 2nd International Meeting on
Respiratory Pathogens. Singapore, Mar. 7-9, 2018.
34. Rossignol J F. J Infect Public Health 2016; 9:227-30.
35. Rossignol J F. Antiviral Res 2014; 110:94-103.
36. Rossignol J F, et al. J Biol Chem 2009; 284:29798-808.
37. Sag D, et al. J Immunol 2008; 181:8633-41.
38. Sleeman K, et al. Antimicrob Agents Chemother 2014; 58:2045-51.
39. TAMIFLU (oseltamivir) prescribing information. Genentech, Inc., South San Francisco, CA, August 2019.
40. Thompson W W, et al. JAMA 2003; 289:179-86.
41. Tilmanis D, et al. Antivir Res 2017; 147:142-48.
42. Troy N M, Bosco A. Resp Res 2016; 17:156.
43. Turner R B. Ann Allergy Asthma Immunol 1997; 78:531-40.
44. United States Department of Labor—Bureau of Labor Statistics. CPI Inflation Calculator. Available at: https://www.bls.gov/data/inflation_calculator.htm
45. Ursic T, et al. BMC Infect Dis 2016; 16:637.
46. Uyeki T M, et al. Clin Infect Dis 2019; 68:e1-47.
47. Wang M, et al. Cell Res 2020; 30:269-71.
48. Wang W, et al. J Biol Chem 2003; 278:27016-23.
49. Worrall G. Can Fam Physician 2011; 57:1289-90.
50. Yu J, et al. Value Health 2020; 23:227-35.

TABLE 4
Abbreviations

| | |
|---|---|
| AE | Adverse Event |
| ALT | Alanine Aminotransferase |
| ARI | Acute Respiratory Illness |
| AST | Aspartate Aminotransferase |
| ATP | Adenosine Triphosphate |
| b.i.d. | Twice Daily |
| BUN | Blood Urea Nitrogen |
| CMH | Cochran-Mantel-Haenszel |
| COPD | Chronic Obstructive Pulmonary Disease |
| COVID-19 | Coronavirus Disease 2019 (caused by SARS-CoV-2) |
| CRF | Case Report Form |
| EC50 | 50% Effective Concentration |
| EDC | Electronic Data Collection |
| EV/RV | Rhinovirus/Enteroviruses |
| FDA | Food and Drug Administration |
| FLU-PRO | InFLUenza Patient-Reported Outcome Questionnaire |
| GCP | Good Clinical Practice |
| GGT | Gamma Glutamyl Transferase |
| HDL | High Density Lipoprotein |
| HIV | Human Immunodeficiency Virus |
| hMPV | Human Metapneumovirus |
| IC50 | 50% Inhibitory Concentration |
| ICF | Informed Consent Form |
| ICH | International Council for Harmonisation |
| IDMC | Independent Data Monitoring Committee |
| IL | Interleukin |
| IRB | Institutional Review Board |
| LDL | Low Density Lipoprotein |
| MDCK | Madin-Darby Canine Kidney |
| NTZ | Nitazoxanide |
| RSV | Respiratory Syncytial Virus |
| RT-PCR | Reverse Transcription Polymerase Chain Reaction |
| SARS-CoV-2 | Severe Acute Respiratory Syndrome Coronavirus 2 |
| VRI | Viral Respiratory Illness |

### Example 4

### A Randomized, Double-Blind, Placebo-Controlled Trial to Evaluate Efficacy and Safety of Nitazoxanide in the Treatment of Mild or Moderate COVID-19

| Title of Study: | A Phase 3, Randomized, Double-Blind, Placebo Controlled Trial to |
|---|---|
| | Evaluate the Efficacy and Safety of Nitazoxanide in the Treatment of Mild |
| | or Moderate COVID-19 |
| Name of Test Drug: | Nitazoxanide 300 mg Tablets |
| Name of Active | Nitazoxanide |
| Ingredient: | |
| IND Number: | 149,166 |

OBJECTIVES

| | | |
|---|---|---|
| Primary Objective: | | To evaluate the effect of NTZ in reducing the time to Sustained Response |
| | | compared to placebo in subjects with mild or moderate COVID-19 |
| Secondary Objectives: | | To evaluate the effect of NTZ in reducing the rate of progression to Severe |
| | | COVID-19 Illness compared to placebo |
| Exploratory Objectives: | | To evaluate: |
| | i. | The proportion of subjects positive for SARS-CoV-2 by TCID₅₀ at each of |
| | | Days 4 and 10 |
| | ii. | The change from baseline in SARS-CoV-2 TCID₅₀ at each of Days 4 and 10 |
| | iii. | The effect of NTZ in reducing the rate of hospitalization compared to placebo |
| | iv. | The effect of NTZ in reducing the rate of mortality compared to placebo |
| Safety Objectives: | | Safety will be assessed by analysis of adverse events. |

METHODOLOGY

| Study Design: | Multicenter, randomized, double-blind trial | |
|---|---|---|
| Treatments: | Subjects be randomized 1:1 to one of the following groups: | |
| | Group 1 (NTZ): | Two NTZ 300 mg tablets b.i.d. for 5 days |
| | | administered orally with food. |
| | Group 2 (Placebo): | Two placebo tablets b.i.d. for 5 days administered |
| | | orally with food. |

| | |
|---|---|
| Study Duration: | Subjects receive 2 tablets b.i.d. for 5 days (10 doses). The follow up |
| | period will extend through the end of study visit at study Day 22. |

SUBJECT POPULATION
Number of Subjects Planned: At least 350 up to 400 subjects with laboratory-confirmed SARS-CoV-2-
infection. This may require enrollment of up to 800 total subjects.
Inclusion Criteria:
Male or female at least 12 years of age
Patient-reported subjective fever within the last 12 hours or oral temperature in-office of at least
99.4° F.
Presence of clinical signs and/or symptoms consistent with worsening or stable mild or moderate
COVID-19 (one of the following is required):
Presence of at least two respiratory symptom domains (head, throat, nose, chest, cough)
with a score of ≥2 as determined by Screening FLU-PRO OR
Presence of at least one respiratory symptom domain (head, throat, nose, chest, cough)
with a score of ≥2 as determined by Screening FLU-PRO with pulse rate ≥90 OR
Presence of at least one respiratory symptom domain (head, throat, nose, chest, cough)
with a score of ≥2 as determined by Screening FLU-PRO with respiratory rate ≥16
AND patient reported assessment that symptoms are present, the symptoms are not consistent
with the subject's usual health, the symptoms interfere with daily activities, and the symptoms
have worsened or remained the same relative to the previous day, as confirmed by responses
to questions in the Screening FLU-PRO.
Onset of symptoms no more than 72 hours before enrollment in the trial. Onset of symptoms is
defined as the earlier of the first time at which the subject experienced subjective fever or
any respiratory symptom (headache/head congestion, throat symptoms, nasal symptoms,
chest symptoms, cough).
Willing and able to provide written informed consent (including assent by legal guardian if under
18 years of age) and comply with the requirements of the protocol, including completion of
the subject diary and all protocol procedures.
Exclusion Criteria:
Persons with any clinical sign or symptoms suggestive of severe systemic illness with COVID-
19, including the following:
shortness of breath at rest,
resting pulse ≥125 beats per minute,
resting respiratory rate ≥30 breaths per minute, or
SpO₂ ≤93% on room air at sea level.
Subjects who experienced a previous episode of acute upper respiratory tract infection, otitis,
bronchitis or sinusitis or received antibiotics for these conditions within two weeks prior to
and including study day 1.
Severely immunodeficient persons including:
Subjects with immunologic disorders or receiving immunosuppressive therapy (e.g., for
organ or bone marrow transplants, immunomodulatory therapies for certain
autoimmune diseases)
Subjects with untreated HIV infection or treated HIV infection with a CD4 count below
350 cells/mm³ in the last six months
Subjects actively undergoing systemic chemotherapy or radiotherapy treatment for
malignancy
Subjects using steroids as maintenance therapy for chronic conditions
Subjects with active respiratory allergies or subjects expected to require anti-allergy medications
during the study period for respiratory allergies.
Females of childbearing potential who are either pregnant or sexually active without the use of
birth control.
Subjects with a history of COVID-19 or known to have developed anti-SARS-CoV-2 antibodies.
Subjects residing in the same household with another subject participating in the study.
Treatment with any investigational drug or vaccine therapy within 30 days prior to screening and
willing to avoid them during the course of the study.
Receipt of any dose of NTZ within seven days prior to screening.
Known sensitivity to NTZ or any of the excipients comprising the study medication.
Subjects unable to swallow oral tablets or capsules.
Subjects with known severe heart, lung, neurological or other systemic disease that the
Investigator believes could preclude safe participation.
Subjects likely or expected to require hospitalization unrelated to COVID-19 during the study
period.
Subjects who, in the judgment of the Investigator, will be unlikely to comply with the
requirements of this protocol including completion of the subject diary.
ASSESSMENTS
Efficacy:
Subjects complete a diary recording oral temperature twice daily and the FLU-PRO © patient
reported outcomes instrument once daily in the evening for 21 days for efficacy evaluation.
Subjects be contacted daily by site staff on study days 2-7 to screen for progression to severe
illness or other complications.
Virology: Nasopharyngeal swab samples be collected on each of study Days 1, 4 and 10 for
analysis of virology endpoints.
Safety: The safety of study drug administration be monitored by assessment of adverse events
from baseline through the Day 22 study exit, clinical laboratory testing at baseline and on Day 10,
physical examinations and vital signs assessments at screening, Day 10 and Day 22 and telephone
monitoring/at home visits during Day 2 through Day 7.
Pharmacokinetics: On the morning of Day 4 prior to dosing, trough plasma samples be
collected.

| | | |
|---|---|---|
| Primary Objective: | | To evaluate the effect of NTZ in reducing the time to Sustained |
| | | Response compared to placebo in subjects with mild or moderate |
| | | COVID-19 |
| Secondary Objective: | | To evaluate the effect of NTZ in reducing the rate of progression to |
| | | Severe COVID-19 Illness compared to placebo |
| Exploratory Objectives: | | To evaluate: |
| | i. | The proportion of subjects positive for SARS-CoV-2 by TCID₅₀ |
| | | at each of Days 4 and 10 |
| | ii. | The change from baseline in SARS-CoV-2 TCID₅₀ at each of |
| | | Days 4 and 10 |
| | iii. | The effect of NTZ in reducing the rate of hospitalization |
| | | compared to placebo |
| | iv. | The effect of NTZ in reducing the rate of mortality compared to |
| | | placebo |
| Safety Objectives: | | Safety be assessed by analysis of adverse events. |

### Study Objectives

### Study Design

### Study Design Overview

This study is a multicenter, randomized, double-blind, placebo-controlled trial designed to evaluate efficacy and safety of NTZ 600 mg administered orally twice daily for five days compared to a placebo for the treatment of mild or moderate COVID-19.

Subjects be stratified according to the following criteria:

| | |
|---|---|
| Severity of COVID-19 Illness: | Mild illness defined as baseline assessments of |
| | (1) at least one respiratory domain* with a |
| | baseline score ≥2 and (2) resting pulse <90 beats |
| | per minute and (3) resting respiratory rate <20 |
| | breaths per minute. |
| | Moderate illness defined as baseline assessments |
| | of (1) at least one respiratory domain* with a |
| | baseline score ≥2 and either (2) resting pulse ≥90 |
| | beats per minute or (3) resting respiratory |
| | rate ≥20 breaths per minute. |
| Time from onset of symptoms to | <36 hours/≥36 hours |
| randomization: | |
| Risk of severe illness (per CDC): | At Increased Risk: Subjects with COPD, Type 2 |
| | diabetes mellitus, obesity (BMI ≥30), chronic |
| | kidney disease, sickle cell disease, or serious |
| | heart conditions (such as heart failure, coronary |
| | artery disease, or cardiomyopathies), asthma |
| | (moderate or severe), cerebrovascular disease, |
| | cystic fibrosis, hypertension or high blood |
| | pressure, immunocompromised state (due to |
| | immune deficiencies, HIV, use of corticosteroids, |
| | or use of other immune-weakening medications), |
| | neurologic conditions (e.g., dementia), liver |
| | disease, pulmonary fibrosis, past or present |
| | history of smoking, thalassemia, or type 1 |
| | diabetes mellitus. Subjects who are ≥65 years of |
| | age. |
| | Not At Increased Risk: Subjects with none of the |
| | above conditions |

*For this purpose, respiratory domains include the following 5 FLU-PRO domains/subdomains: chest, cough, nose, throat and head.

Within strata, subjects will be randomized 1:1 to one of the following groups:
Group 1 (NTZ): Two NTZ 300 mg tablets b.i.d. for 5 days
Group 2 (Placebo): Two placebo tablets b.i.d. for 5 days

### Study Population

### Inclusion Criteria

1 Male or female outpatients at least 12 years of age
2. Subjective fever within the last 12 hours or oral temperature in-office of at least 99.4° F.
3. Presence of clinical signs and/or symptoms consistent with worsening or stable mild or moderate COVID-19 (one of the following is required):
a) Presence of at least two respiratory symptom domains (head, throat, nose, chest, cough) with a score of ≥2 as determined by Screening FLU-PRO OR
b) Presence of at least one respiratory symptom domain (head, throat, nose, chest, cough) with a score of ≥2 as determined by Screening FLU-PRO with pulse rate ≥90 OR
c) Presence of at least one respiratory symptom domain (head, throat, nose, chest, cough) with a score of ≥2 as determined by Screening FLU-PRO with respiratory rate ≥16 AND patient reported assessment that symptoms are present, the symptoms are not consistent with the subject's usual health, the symptoms interfere with daily activities, and the symptoms have worsened or remained the same relative to the previous day, as confirmed by responses to questions in the Screening FLU-PRO.
4. Onset of symptoms no more than 72 hours before enrollment in the trial. Onset of symptoms is defined as the earlier of the first time at which the subject experienced subjective fever or any respiratory symptom (head, throat, nose, chest, or cough symptoms).
5. Willing and able to provide written informed consent (including assent by legal guardian if under 18 years of age) and comply with the requirements of the protocol, including completion of the subject diary and all protocol procedures.

### Exclusion Criteria

1. Persons with any clinical sign or symptoms suggestive of severe systemic illness with COVID-19, including the following:
a) shortness of breath at rest,
b) resting pulse ≥125 beats per minute,
c) resting respiratory rate ≥30 breaths per minute, or
d) SpO₂≤93% on room air at sea level.
2. Subjects who experienced a previous episode of acute upper respiratory tract infection, otitis, bronchitis or sinusitis or received antibiotics for these conditions within two weeks prior to and including study day 1.
3. Severely immunodeficient persons including:
a) Subjects with immunologic disorders or receiving immunosuppressive therapy (e.g., for organ or bone marrow transplants, immunomodulatory therapies for certain autoimmune diseases)
b) Subjects with untreated HIV infection or treated HIV infection with a CD4 count below 350 cells/mm³ in the last six months
c) Subjects actively undergoing systemic chemotherapy or radiotherapy treatment for malignancy
d) Subjects using steroids as maintenance therapy for chronic conditions
4. Subjects with active respiratory allergies or subjects expected to require anti-allergy medications during the study period for respiratory allergies.
5. Females of childbearing potential who are either pregnant or sexually active without the use of birth control. Female subjects of child-bearing potential that are sexually active must have a negative baseline pregnancy test and must agree to continue an acceptable method of birth control for the duration of the study and for 1 month post-treatment. A double barrier method, oral birth control pills administered for at least 2 monthly cycles prior to study drug administration, an IUD, or medroxyprogesterone acetate administered intramuscularly for a minimum of one month prior to study drug administration are acceptable methods of birth control for inclusion into the study. Female subjects are considered of childbearing potential unless they are postmenopausal (absence of menstrual bleeding for 1 year—or 6 months if laboratory confirmation of hormonal status), or have had a hysterectomy, bilateral tubular ligation or bilateral oophorectomy.
6. Subjects with a history of COVID-19 or known to have developed anti-SARS-CoV-2 antibodies.
7. Subjects residing in the same household with another subject participating in the study.
8. Treatment with any investigational drug or vaccine therapy within 30 days prior to screening and willing to avoid them during the course of the study.
9. Receipt of any dose of NTZ within seven days prior to screening.
10. Known sensitivity to NTZ or any of the excipients comprising the study medication.
11. Subjects unable to swallow oral tablets or capsules.
12. Subjects with known severe heart, lung, neurological or other systemic disease that the Investigator believes could preclude safe participation.
13. Subjects likely or expected to require hospitalization unrelated to COVID-19 during the study period.
14. Subjects who, in the judgment of the Investigator, will be unlikely to comply with the requirements of this protocol including completion of the subject diary

### Dosing and Administration

Group 1 (NTZ): Subjects will receive two NTZ 300 mg tablets b.i.d. with food (<1 hour after food intake) and a B complex vitamin (Super B-Complex™, Igennus Healthcare Nutrition, Cambridge, UK) b.i.d. for 5 days
Group 2 (Placebo): Subjects will receive two placebo tablets b.i.d. with food (<1 hour after food intake) and a B complex vitamin (Super B-Complex™, Igennus Healthcare Nutrition, Cambridge, UK) b.i.d. for 5 days

The food prior to drug intake should preferably be a high-fat meal, but at minimum a cereal bar.

All subjects will receive a vitamin B complex supplement one tablet twice a day to help mask any potential chromaturia attributed to NTZ and aid in maintaining study blinding.

### Diagnostic Virology Testing

Diagnostic virology testing will be performed using nasopharyngeal swab samples collected at the Baseline, Day 4 and Day 10.

The ePlex® Respiratory Pathogen Panel (GenMark, Carlsbad, CA) will be used to detect influenza A (non-specific as to subtype); influenza A H1, H1N1 (2009), H3 subtypes; influenza B; RSV A and B; parainfluenza 1, 2, 3 and 4; hMPV; adenovirus; human EV/RV; coronavirus NL63, HKU1, 229E and OC43; Chlamydophila pneumoniae; and Mycoplasma pneumoniae.

The Aptima® SARS-CoV-2 Assay (Hologic, Inc., San Diego, CA) will be used to detect SARS-CoV-2.

All Baseline nasopharyngeal swabs will be tested by both PCR assays. If the Baseline nasopharyngeal swab is positive for SARS-CoV-2, the Day 4 and 10 sample will be tested for SARS-CoV-2.

### Quantitative Virology Testing

Nasopharyngeal swab samples testing positive for SARS-CoV-2 will be subjected to TCID₅₀ for analysis of quantitative changes in viral load.

### Serology Testing

Baseline and Day 22 blood samples will be tested for quantitative anti-SARS-CoV-2 antibodies.

### Plan for Monitoring Viral Resistance

If any Day 10 nasopharyngeal swab sample is positive for SARS-CoV-2 by the SARS-CoV-2 assay, the Baseline and Day 10 nasopharyngeal swab samples for the subject will be tested for post-treatment reduced susceptibility to tizoxanide.

| | | |
|---|---|---|
| Primary Efficacy Parameter: | | Time from first dose to Sustained Response |
| | | based on the FLU-PRO Patient Reported |
| | | Outcomes Instrument. |
| Secondary Efficacy Parameter: | | Proportion of subjects progressing to severe |
| | | COVID-19 illness. |
| Exploratory Efficacy Parameters: | i. | Proportion of subjects positive for SARS- |
| | | CoV-2 by culture at Days 4 and 10. |
| | ii. | Mean changes from baseline in SARS- |
| | | CoV-2 TCID₅₀. |
| | iii. | Proportion of subjects hospitalized due to |
| | | COVID-19 or complications thereof. |
| | iv. | Proportion of subjects with mortality due |
| | | to COVID-19 or complications thereof. |

Response Definitions

| Sustained Response: | A decrease in total FLU-PRO score from the | |
|---|---|---|
| | previous diary with patient assessment that | |
| | symptoms are at least “somewhat better than | |
| | yesterday”, no oral temperature ≥100.4° F. in the | |
| | prior 24 hours, and no future increase in any of | |
| | the FLU-PRO domains except within the following levels: | |
| | FLU-PRO Domain* | Background Level |
| | Body/Systemic | 0.56 |
| | Throat | 0.67 |
| | Eyes | 0.67 |
| | Gastrointestinal | 2.00 |
| | Head | 2.00 |
| | Nose | Score at time of |
| | | response or 0.75, |
| | | whichever is greater |
| | Chest | Score at time of |
| | | response |
| | Cough | Score at time of |
| | | response or 1.75, |
| | | whichever is greater |

| | |
|---|---|
| Time of Sustained Response: | The time of the first diary at which all Sustained |
| | Response criteria are met. |
| Time to Sustained Response: | The time (hours) from the first dose of study |
| | medication and the Time of Sustained |
| | Response. |
| Sever COVID-19 Illness: | Subject must have (1) shortness of breath at rest |
| | and (2) SpO₂ ≤93% on room air or PaO₂/FiO₂ <300. |

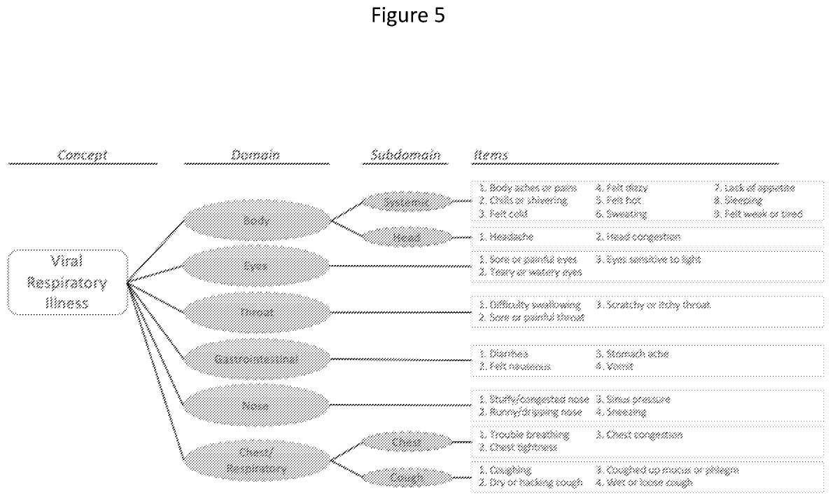

*A diagram of the FLU-PRO domain/subdomain structure is in FIG. 5.

### Efficacy Variables

### Statistical Methodology

### Efficacy Analyses

Efficacy analyses will be based on a population consisting of all subjects randomized who receive at least one dose of study medication and are positive for SARS-CoV-2 by PCR at Baseline. For time-to-event analyses, a test of significance (as described in the following) will be performed with descriptive statistics provided, including the use of Kaplan-Meier figures.

There will be one primary efficacy analysis:
Time to Sustained Response for the NTZ treatment group will be compared to that of the placebo treatment group using a stratified Gehan-Wilcoxon test (α=0.05) where stratification will follow that used for randomization. Subjects without a Sustained Response recorded will be treated as censored as of the last diary without a documented Sustained Response.

If the primary analysis is significant at the 0.05 level, the key secondary efficacy analysis will be formally evaluated at the 0.05 level as follows:
Proportions of subjects progressing to Severe COVID-19 Illness will be compared between the treatment groups using a Cochran-Mantel-Haenszel (CMH) test stratified by the randomization strata.

Exploratory analyses will be performed as follows:
Proportions of subjects positive for SARS-CoV-2 by culture (TCID₅₀) at study Day 4 and Day 10 will be compared between the treatment groups using a Cochran-Mantel-Haenszel (CMH) test stratified by the randomization strata.
Mean changes from baseline in SARS-CoV-2 TCID₅₀ at Day 4 and Day 10 will be compared using a t-test.
Proportions of subjects hospitalized due to COVID-19 or complications thereof will be compared across the treatment groups using a CMH test stratified by the randomization strata.
Proportions of subjects with mortality related to COVID-19 or complications thereof will be compared across the treatment groups using a CMH test stratified by the randomization strata.

### Population Pharmacokinetics Analysis

On Day 4, plasma samples will be collected before the morning dose (at the trough) for determination of drug concentration. These data will allow for analysis of relationships between trough plasma concentrations and clinical and virologic response.

Trough plasma concentrations of tizoxanide and tizoxanide glucuronide will be summarized descriptively for NTZ-treated subjects. Exploratory analyses will be conducted to evaluate the relationships between plasma concentrations and age, race, gender, body weight, body mass index, VRI and adverse events.

### Safety Analyses

All randomized subjects who receive at least one dose of study medication will be evaluated for drug safety. Safety analyses will be done descriptively.

### REFERENCES

1 ALINIA (nitazoxanide) prescribing information. Romark, L. C., Tampa, FL, June 2019.
2. Anderson V R, Curran M P. Drugs 2007; 67:1947-67.
3. Ashton L V, et al. Vet Med Int 2010; 2010:891010.
4. Braakman I, Helenius J, Helenius A. Role of ATP and disulphide bonds during protein folding in the endoplasmic reticulum. Nature 1992; 356:260-62.
5. Braakman I, et al. J Cell Biol 1991; 114:401-11.
6. Cao J, et al Res 2015; 114:1-10.
7. Centers for Disease Control and Prevention. Coronavirus Disease 2019 (COVID-19): COVIDView—Key Updates for Week 22, ending May 30, 2020.
8. Centers for Disease Control and Prevention. Coronavirus Disease 2019 (COVID-19): Symptoms of Coronavirus. Accessed 12 Jun. 2020. Available at: https://www.cdc.gov/coronavirus/2019-ncov/symptoms-testing/symptoms.html
9. Chang C W, et al J Biomed Sci 2009; 16:80.
10. Chen L, et al. Clinical Characteristics of Pregnant Women with COVID-19 in Wuhan, China. NEJM 2020; 382:e100.
11. Doms R W, et al. J Cell Biol 1987; 105:1957-69.
12. Goldberg B. New York survey suggests nearly 14% in state may have coronavirus antibodies. Reuters. 23 Apr. 2020. Available at: https://www.reuters.com/article/us-health-coronavirus-usa-new-york/new-york-survey-suggests-nearly-14-in-state-may-have-coronavirus-antibodies-idUSKCN2252WN?il=0
13. Goldman J D et al. Remdesivir for 5 or 10 Day s in Patients with Severe COVID-19. NEJM 2020. Published online 27 May 2020. Available at: https://www.nejm.org/doi/ful1/10.1056/NEJMoa2015301
14. Hadad G M, et al. J Chromatogr Sci. 2012; 50:509-15.
15. Hong S K, et al. Int Immunopharmacol 2012; 13:23-27.
16. Johns Hopkins University and Medicine COVID-19 map. Johns Hopkins Coronavirus Resource Center. Accessed: 12 Jun. 2020. Available at: https://coronavirus.jhu.edu/map.html
17. Lee J H, et al. Int J Obes 2017; 41:645-51.
18. Li, X, Ma, X. Crit Care 24, 198 (2020). https://doi.org/10.1186/s13054-020-02911-9
19. Mirazimi A, Svensson L. J Virol 2000; 74:8048-52.
20. OECD. OECD Economic Outlook, June 2020—The world economy on a tightrope. Accessed 12 Jun. 2020. Available at: http://www.oecd.org/economic-outlook/june-2020/21.
21. Piacentini S, et al. Sci Rep 2018; 8:10425.
22. Powers J H, et al. PLoS ONE 2018 13(3):d0194180.
23. Rossignol J F, et al J Biol Chem 2009; 284:29798-808.
24. Rossignol J F, van Baalen C. Presented at the 2nd International Meeting on Respiratory Pathogens. Singapore, Mar. 7-9, 2018.
25. Rossignol J F. J Infect Public Health 2016; 9:227-30.
26. Rossignol J F. Antiviral Res 2014; 110:94-103.
27. Sag D, et al. J Immunol 2008; 181:8633-41.
28. Sleeman K, et al. Antimicrob Agents Chemother 2014; 58:2045-51.
29. Sood N, Simon P, Ebner P. Seroprevalence of SARS-CoV-2-Specific Antibodies Among Adults in Los Angeles County, California, on Apr. 10-11, 2020. JAMA 2020. Published online May 18, 2020. Available at: https://jamanetwork.com/journals/jama/fullarticle/2766367
30. St. Jude Children's Research Hospital. Infections in Immunocompromised Patients. Accessed 22 Jun. 2020. Available at: https://www.stjude.org/treatment/patient-resources/caregiver-resources/infection-tips/infections-immunocompromised-patients.html
31. Tilmanis D, et al. Antivir Res 2017; 147:142-48.
32. van der Vries E, et al. PLoS Pathog. 2013; 9(5):e1003343.
33. Wang M, et al. Cell Res 2020; 30:269-71.
34. Wang W, et al. J Biol Chem 2003; 278:27016-23.
35. Wolfel, R., et al. Nature 581, 465-469 (2020).
36. Yu J, et al. Value Health 2020; 23:227-35.
37. Zhu N, et al. NEJM 2020; 382:727-733.

### Example 5

### Early Treatment with Nitazoxanide Prevents Worsening of Mild and Moderate COVID-19 and Subsequent Hospitalization

### Summary

There is a need for treatment that can prevent the progression to severe COVID-19 and hospitalization for patients with mild or moderate COVID-19

Methods: A randomized double-blind placebo-controlled clinical trial in 36 centers in the U.S. and Puerto Rico investigated the safety and effectiveness of oral nitazoxanide 600 mg twice daily for 5 days in outpatients with symptoms of mild or moderate COVID-19 enrolled within 72 hours of symptom onset. Key objectives were reduction of duration of symptoms (primary) and progression to severe illness (key secondary).

Results: 1,092 subjects were enrolled, and 379 with laboratory-confirmed SARS-CoV-2 infection were analyzed. Overall, times to sustained clinical recovery were similar for the two arms. Nitazoxanide treatment was associated with an 85% reduction in the progression to severe COVID-19 (1/184, [0.5%] vs. 7/195, [3.6%], p=0.07) and 82% reduction in the rate of hospitalization, emergency room visit or death (1/184 [0.5%] vs. 6/195 [3.1%], p=0.12). In subjects with mild illness at baseline, treatment was also associated with a 3.1-day reduction in median time to sustained clinical recovery and a 5.2-day reduction in time to return to usual health. Nitazoxanide was safe and well tolerated.

Conclusions: Treatment of mild or moderate COVID-19 with a five-day course of oral nitazoxanide was safe and well tolerated and was associated with an 85% reduction in the progression to severe illness and a 3- to 5-day reduction of the duration of mild illness.

### Introduction

The progress in developing effective vaccines against SARS-CoV-2 infection has been remarkable. Coupling effective therapies with vaccination programs may be important for controlling the pandemic.¹ However, the outcome of significant efforts to develop novel therapies, particularly in outpatients, has so far been limited. Promising results have been achieved with monoclonal antibodies, yet these treatments are costly, prone to viral resistance, and can only be administered in a clinic setting²⁻⁴. Hence, there is still a need for medications which can be distributed broadly and in settings with scarce healthcare resources to prevent the worsening of mild or moderate COVID-19 symptoms and subsequent hospitalization.

Nitazoxanide is approved for use in the United States for the treatment of diarrhea caused by Cryptosporidium parvum and Giardia intestinalis infections and has been used throughout Latin America and Asia for the treatment of intestinal parasitic infections. In the 25 years since nitazoxanide was first introduced, approximately 500 million people have been treated worldwide, and the drug has demonstrated a favorable safety record in both adults and children.

The present multicenter, randomized, double-blind, placebo-controlled trial provides evidence nitazoxanide prevents the progression to severe illness and hospitalization and reduces the duration of mild illness when administered to patients within 72 hours of symptomatic SARS-CoV-2 infection.

### Methods

### Study Design and Subjects

This was a randomized double-blind placebo-controlled trial conducted in 36 outpatient medical clinics in the U.S. and Puerto Rico in accordance with current good clinical practices and applicable regulations.

Subjects at least 12 years of age presenting within 72 hours of onset of symptoms of mild or moderate COVID-19 were eligible to participate in the trial. Minimum symptom requirements were: at least two respiratory symptom domains (head, throat, nose, chest, cough) with a score of ≥2 as determined by scoring the InFLUenza Patient-Reported Outcomes (FLU-PRO©)²¹ questionnaire administered at screening (only one domain score required to be ≥2 if pulse rate ≥90 beats per minute or respiratory rate ≥16 breaths per minute), with no improvement in overall symptom severity from the prior day. Key exclusion criteria were: (i) signs or symptoms suggestive of severe COVID-19 including shortness of breath at rest, resting pulse rate ≥125 beats per minute, resting respiratory rate ≥30 breaths per minute, or SpO₂≤93% on room air at sea level; (ii) previous COVID-19 infection, (iii) immunodeficiency; and (iv) pregnant females and sexually active females of childbearing potential not using birth control.

Nitazoxanide was administered as two 300 mg extended release tablets (600 mg per dose) orally with food twice daily for five days.

### Randomization and Masking

Eligible subjects were centrally randomized using an interactive web response system 1:1 to receive treatment with nitazoxanide or matching placebo tablets. In addition to study medication, all subjects received a vitamin B complex supplement (Super B-Complex™, Igennus Healthcare Nutrition, Cambridge, UK) twice daily to mask any potential chromaturia attributed to nitazoxanide. The randomization list was masked to study participants, the sponsor, investigators, study monitors, and laboratory personnel until the database was locked.

Randomization was stratified according to the severity of COVID-19 illness at baseline (mild or moderate), time from onset of symptoms (<36 hours or ≥36 hours), and whether subjects had risk factors for severe illness based on CDC criteria current at the initiation of the study (see the Supplementary Material infra for CDC criteria). Moderate illness was defined by resting pulse ≥90 beats per minute and/or resting respiratory rate ≥20 breaths per minute.

### Study Procedures

After randomization, eligible subjects underwent a physical examination, collection of nasopharyngeal swabs, and blood and urine samples for laboratory safety testing and assessment of SARS-CoV-2 antibody titers. Study drug was dispensed, and subjects were followed for 28 days. Subjects were instructed to complete electronic diaries recording oral temperature twice daily and symptom severity once daily in the evening for 21 days and were contacted daily by telephone by site staff on study days 2-7 and 28 to verify compliance and screen for progression to severe illness or other complications. Repeat nasopharyngeal swabs were collected on study days 4 and 10. Follow up blood and urine samples for laboratory safety testing and assessment of SARS-CoV-2 antibody titers were collected on study day 22. Subjects were allowed to use acetaminophen and/or dextromethorphan for symptom relief along with standard-of-care rescue medications. Adverse events were collected continuously throughout the study and monitored until the events resolved.

In the absence of any patient-reported outcomes instrument validated specifically for collecting data to measure symptoms of COVID-19, symptom data was collected using the FLU-PRO Plus® symptoms questionnaire. The InFLUenza Patient-Reported Outcome Questionnaire (FLUPRO©) was developed in accordance with psychometric best practices and FDA guidance for the measurement of symptoms of influenza²¹. Subsequent literature searches and clinical data analyses support the content and construct validity of the instrument for illness caused by non-influenza respiratory viruses including adenovirus, endemic coronaviruses, enteroviruses including rhinoviruses, parainfluenza and respiratory syncytial virus. The questionnaire was completed using an electronic diary app downloaded to each subject's smart phone or a provisioned electronic device so that diary entries were time stamped to ensure timely recording, thereby mitigating risks of recall bias.

Nasopharyngeal swab samples collected at baseline, day 4 and day 10 were tested using the Aptima® SARS-CoV-2 assay (Hologic, Inc., San Diego, CA) and ePlex® Respiratory Pathogen Panel (“ePlex RPP”, GenMark, Carlsbad, California). Baseline, day 4 and day 10 nasopharyngeal swab samples positive for SARS-CoV-2 by the Aptima® SARS-CoV-2 assay were subjected to RT-PCR for analysis of quantitative changes in viral load. Blood samples collected at baseline and day 22 were tested for quantitative anti-SARS-CoV-2 antibodies.

### Primary and Secondary Outcomes

The primary endpoint was time from the first dose to sustained response (TSR), a measure of meaningful within-subject symptom improvement developed and validated in subjects with influenza infection. The performance characteristics of the FLU-PRO instrument and appropriateness of background levels in subjects with SARS-CoV-2 infection were confirmed by blinded analysis of diary data for this study after database lock and prior to unblinding.

The key secondary endpoint was the rate of progression to severe COVID-19 illness (shortness of breath at rest and SpO₂≤93% on room air or PaO₂/FiO₂<300). This definition was selected over a definition including hospitalization due to variability in physician decisions regarding hospital admission.

### Statistical Analysis

Efficacy analyses were based on a modified intention to treat (mITT) population of subjects testing positive for SARS-CoV-2 at baseline. All subjects receiving at least one dose of study medication were included in the safety analyses.

In the primary analysis, TSR for the nitazoxanide treatment group was compared to that of the placebo treatment group using a stratified Gehan-Wilcoxon test (α=0.05) where stratification followed that used for randomization. Subjects without a sustained response recorded were treated as censored as of the last diary completed, except for subjects who were hospitalized or died during the study, who were censored at Day 21.

In the absence of prior experience in subjects with COVID-19, the sample size was determined based upon data from two prior clinical trials of nitazoxanide in subjects with viral respiratory illnesses caused by influenza or rhinoviruses. A sample size of 312 subjects (156 per group) was calculated to provide 90% power to detect a statistically significant difference in the survival distributions between the nitazoxanide and placebo groups (Gehan rank test, two-sided α=0.05).

In the key secondary analysis, proportions of subjects progressing to severe COVID-19 illness were compared between the treatment groups using a Cochran-Mantel-Haenszel (CMH) test stratified by the randomization strata.

### Results

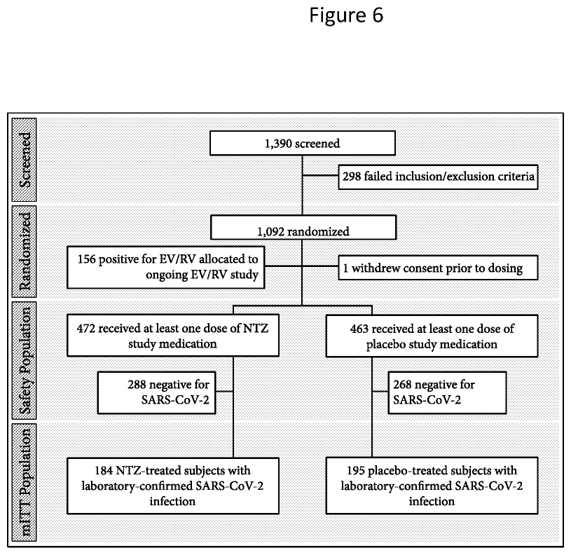

1,092 subjects were enrolled at 36 sites in the U.S. and Puerto Rico, including 379 with laboratory-confirmed SARS-CoV-2 infection (FIG. 6). Demographic and disease-related characteristics of the SARS-CoV-2-infected population are summarized in Table 5.

TABLE 5
Summary of Baseline Demographic and Disease-Related Characteristics, ITTI Population

| | | | | | All mITT | |
|---|---|---|---|---|---|---|
| | Nitazoxanide | | Placebo | | Subjects | |
| | (N = 184) | | (N = 195) | | (N = 379) | |
| Male, N (%) | 83 | (45.1%) | 82 | (42.1%) | 165 | (43.5%) |
| Median (Range) Age (years) | 38 | (12-83) | 42 | (13-81) | 40 | (12-83) |
| Race or Ethnic Group, N (%) | | | | | | |
| White | 117 | (63.6%) | 116 | (59.5%) | 233 | (61.5%) |
| Hispanic or Latino | 59 | (32.1%) | 71 | (36.4%) | 130 | (34.3%) |
| Black or African American | 4 | (2.2%) | 4 | (2.1%) | 8 | (2.1%) |
| Asian | 2 | (1.1%) | 4 | (2.1%) | 6 | (1.6%) |
| Other | 2 | (1.1%) | 0 | (0.0%) | 2 | (0.5%) |
| Median (IQR) BMI | 28.7 | (25.3, 33.5) | 29.1 | (25.7, 33.6) | 28.9 | (25.5, 33.5) |
| Median Hours Since Onset of | 43.9 | | 46.5 | | 45.9 | |
| Symptoms | | | | | | |
| Moderate Disease Severity, N (%) | 68 | (37.0%) | 65 | (33.3%) | 133 | (35.2%) |
| At Risk of Severe Illness per CDC | 112 | (60.9%) | 126 | (64.6%) | 238 | (62.8%) |
| Guidelines, N (%) | | | | | | |
| Viral Load, log₁₀ RNA copies/mL | | | | | | |
| Median (IQR) | 6.38 | (4.71, 7.40¹) | 6.34 | (4.40, 7.40¹) | 6.36 | (4.62, 7.40¹) |
| SARS-COV-2 Antibody-Positive, | 18 | (4.7%) | 20 | (5.3%) | 38 | (10.0%) |
| N (%) | | | | | | |

¹7.40 log10 copies/mL is the upper limit of quantitation of the assay.

Primary Outcome. Ninety-two percent (92%) of all possible daily FLU-PRO questionnaires were completed, and only 22 (5.8%) subjects were censored in the primary analysis due to missing diary data prior to the day 22 visit. Median (IQR) TSR were 13.28 (6.26->21) and 12.35 (7.18->21) days for the nitazoxanide and placebo groups, respectively (p=0.88).
Key Secondary Outcome. Eight subjects met the criteria for progression to severe COVID-19, Table 6. Treatment with nitazoxanide was associated with an 85% reduction of progression to severe illness compared to placebo (1/184 [0.5%] for the nitazoxanide group compared to 7/195 [3.6%] for the placebo group, p=0.07), Table 7.

[symbol]

6: Subjects Progressing to Severe COVID-19 Illness

| | | | | | | | Medical | | |
|---|---|---|---|---|---|---|---|---|---|
| [symbol] ent | Age | | | Viral Load¹ | | Study | Attention | Secondary | |
| [symbol] | Group | Sex | BMI | Day 1/Day 4 | Comorbidities | Day | Required | Diagnosis | [symbol] |
| Placebo | 70-80 | M | 31.7 | 6.18/≥7.40 | Type 2 diabetes mellitus, Parkinson's | 7 | Hospitalized | COVID-19 | [symbol] |
| | | | | | disease, neuropathy, colon cancer/colon | | | pneumonia | |
| | | | | | re-sectioning, benign prostatic | | | | |
| | | | | | hyperplasia, obesity | | | | |
| NTZ | 70-80 | M | 25.5 | 7.39/4.79 | Hypertension, coronary artery disease, | 5 | Hospitalized | COVID-19 | [symbol] |
| | | | | | atrial fibrillation, bilateral iliac stents, | | | pneumonia | |
| | | | | | type 2 diabetes mellitus, peripheral | | | | |
| | | | | | vascular disease, hyperlipidemia, | | | | |
| | | | | | decreased renal function, cholecystectomy, | | | | |
| | | | | | prostate cancer with prostatectomy, | | | | |
| | | | | | hypogonadism, gout | | | | |
| Placebo | 50-60 | F | 22.8 | 5.96/≥7.40 | Hypertension, anxiety, migraine | 3 | Hospitalized | Syncope | 93% |
| | | | | | headaches, cigarette use | | | | |
| Placebo | 60-70 | F | 36.4 | 6.54/7.06 | Hyperlipidemia, osteoarthritis, | 10 | Emergency | COVID-19 | 93% |
| | | | | | insomnia, anxiety, obesity | | room | pneumonia | |
| Placebo | 60-70 | M | 33.0 | ND/ND | Hypertension, hypercholesterolemia, | 7 | Hospitalized | Dyspnea | 88% |
| | | | | | pre-diabetes, gastroesophageal reflux | | | | |
| | | | | | disease, osteoarthritis, anxiety, | | | | |
| | | | | | prostate cancer and surgery, bilateral | | | | |
| | | | | | hip arthroplasty, cataracts, obesity | | | | |
| Placebo | 50-60 | M | 26.8 | ≥7.40/NC | Type 2 diabetes mellitus, borderline | 9 | Hospitalized | COVID-19 | 84% |
| | | | | | reduced kidney function, coronary artery | | | pneumonia | |
| | | | | | disease, coronary artery bypass graft, | | | | |
| | | | | | neuropathy, hypothyroidism, hernias | | | | |
| Placebo | 30-40 | M | 35.4 | ≥7.40/IND | Essential hypertension, elevated liver | 5 | Clinic visit | Asthma | 93% |
| | | | | | enzymes, asthma, obesity | | | (exacerbation) | |
| Placebo | 50-60 | M | 34.9 | 6.47/ND | Hypertension, elevated liver enzymes, | 7 | Hospitalized | COVID-19 | 90% |
| | | | | | previous stroke, subcarinal adenopathy, | | | pneumonia | |
| | | | | | migraines, nerve issues due to injury, | | | | |
| | | | | | obesity | | | | |

¹log₁₀ RNA copies/mL, upper limit of quantitation of the assay was 7.40 log₁₀ RNA copies/mL
²ND = Not Done due to insufficient sample; NC = Sample Not Collected (visit missed), IND = Indeterminate result by RT-PCR [symbol]
indicates data missing or illegible when filed

TABLE 7
Analyses of the Subjects Progressing to Severe COVID-19
with Subgroups Based Upon Different At-Risk Definitions

| | | | Relative |
|---|---|---|---|
| | Placebo | NTZ | Reduction |
| All SARS-CoV-2-Positive Subjects | 7/195 | 1/184 | 85% |
| | (3.6%) | (0.5%) | |
| Subgroups: | | | |
| “May Be” or “At | 7/126 | 1/112 | 84% |
| Increased Risk” for Severe | (5.6%) | (0.9%) | |
| COVID-19 Illness per CDC (protocol- | | | |
| defined stratification factor) | | | |
| At Increased Risk for Severe | 7/121 | 1/104 | 83% |
| COVID-19 Illness per CDC | (5.8%) | (1.0%) | |
| At High Risk of Progressing to | 6/69 | 1/60 | 81% |
| Severe COVID-19 and/or Hospitalization | (8.7%) | (1.7%) | |
| (per EUA documents for | | | |
| monoclonal antibodies)¹ | | | |

¹≥65 years of age, BMI ≥35 kg/m2, chronic kidney disease, diabetes, immunosuppressive disease, current receipt of immunosuppressive treatment, or ≥55 years of age with at least one of cardiovascular disease, hypertension, or chronic obstructive pulmonary disease or another chronic respiratory disease

Exploratory Outcomes. Treatment with nitazoxanide was associated with an 82% reduction in the rate of hospitalization, emergency room visit or death (p=0.11), Table 8.

TABLE 8
Analyses of the Subjects Experiencing Hospitalization,
Emergency Room Visit or Death with Subgroups Based
Upon Different At-Risk Definitions

| | | | Relative |
|---|---|---|---|
| | Placebo | NTZ | Reduction |
| All SARS-CoV-2-Positive Subjects | 6/195 | 1/184 | 82% |
| | (3.1%) | (0.5%) | |
| Subgroups: | | | |
| “May Be” or “At | 6/126 | 1/112 | 81% |
| Increased Risk” for Severe | (5.6%) | (0.9%) | |
| COVID-19 Illness per CDC (protocol- | | | |
| defined stratification factor) | | | |
| At Increased Risk for Severe | 6/121 | 1/104 | 81% |
| COVID-19 Illness per CDC | (5.8%) | (1.0%) | |
| At High Risk of Progressing to | 5/69 | 1/60 | 77% |
| Severe COVID-19 and/or | (8.7%) | (1.7%) | |
| Hospitalization (per EUA | | | |
| documents for monoclonal antibodies)¹ | | | |

¹≥65 years of age, BMI ≥35 kg/m2, chronic kidney disease, diabetes, immunosuppressive disease, current receipt of immunosuppressive treatment, or ≥55 years of age with at least one of cardiovascular disease, hypertension, or chronic obstructive pulmonary disease or another chronic respiratory disease

Ninety four percent (94%) and 70% of subjects tested positive for SARS-CoV-2 RNA in nasopharyngeal swabs collected at study days 4 and 10, respectively. Qualitative and quantitative tests to detect SARS-CoV-2 were not significantly different between the treatment arms at these time points, Table 9.

TABLE 9
Virologic Data by Time Point

| | NTZ | | | Placebo | | |
|---|---|---|---|---|---|---|
| | | | Mean (SD) Viral Load | | | Mean (SD) Viral Load |
| | N Samples | Percent | Change from Baseline | N Samples | Percent | Change from Baseline |
| Time Point | Analyzed | Positive | (log₁₀ copies/mL) | Analyzed | Positive | (log₁₀ copies/mL) |
| Day 4 | 177 | 94% | −0.70 (1.573) | 178 | 94% | −1.02 (1.668) |
| Day 10 | 177 | 73% | −2.49 (1.582) | 182 | 71% | −2.61 (1.604) |

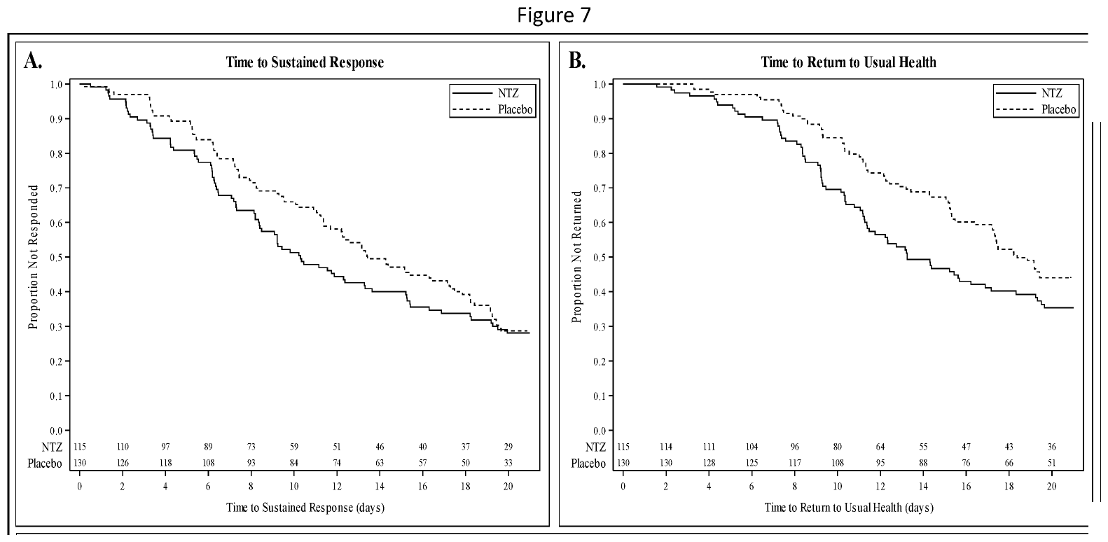

In the 246 subjects (65%) with mild illness at baseline, treatment with nitazoxanide was associated with a 3.1-day reduction of median TSR (median [IQR]=10.3 days [6.2->21] for nitazoxanide [n=116] compared to 13.4 days [7.4->21] for the placebo group [n=130], p=0.0932) and a 5.2-day reduction of median time from first dose until the subjects reported returning to usual health (median [IQR]=13.2 days [9.2->21] for nitazoxanide compared to 18.4 days [11.4->21] for the placebo group, p=0.0075), FIG. 7. In the 133 subjects (35%) with moderate illness at baseline, treatment with nitazoxanide was associated with a longer TSR and time to return to usual health. None of the 68 subjects with moderate illness in the nitazoxanide arm experienced progression to severe illness while two of 65 in the placebo arm did.

Safety. Nitazoxanide was safe and well tolerated. Sixty-three subjects (13.3%) in the nitazoxanide group and 75 (16.2%) in the placebo group reported at least one adverse event, predominately classified as mild or moderate in severity and unrelated or possibly related to study drug. Only diarrhea was reported in ≥2% in any treatment group (n=16 [3.4%] in the nitazoxanide group and n=10 [2.2%] in the placebo group). Frequency, severity and assessment of relationship to study drug of adverse events were similar across treatment groups. Two subjects treated with nitazoxanide and seven receiving placebo reported serious adverse events, all unrelated to the study drug. Two subjects (both in the nitazoxanide treatment group) died during the study, one due to severe COVID-19 and the other (SARS-CoV-2 negative) secondary to aspiration 19 days after completing therapy. Neither event was considered related to study medication. Two nitazoxanide-treated subjects and three receiving placebo discontinued study medication due to adverse events.

### Discussion

The COVID-19 pandemic continues with surges caused by SARS-CoV-2 variants, and the rate of hospitalization poses a major threat to health care systems in many countries. Despite the development of vaccines which are now being distributed, widespread vaccination will need time to be implemented worldwide and will not fully prevent infection. Thus, there is a critical need for a safe, easy-to-administer antiviral therapeutic that can be distributed through pharmacies and administered early for treatment of mild or moderate COVID-19—ideally a host-directed antiviral with a high barrier to resistance that could provide a line of defense against emerging variants.

A multicenter randomized double-blind placebo-controlled trial conducted at 36 outpatient centers in the United States and Puerto Rico is reported. The study employed a concurrent placebo control and enrolled a broad range of subjects at least 12 years of age, 63% of whom had risk factors placing them at higher risk of severe COVID-19. Subjects were enrolled based upon symptoms to ensure early treatment, avoiding limitations associated with the availability of and delays in diagnostic testing, and 379 subjects with confirmed SARS-CoV-2 infection were analyzed for effectiveness. The trial was appropriately blinded, and subjects were closely followed for 28 days. The trial was designed early during the course of the pandemic without the benefit of prior experience with COVID-19, nevertheless, the endpoints were objective, relevant and well-defined, and rigorous data collection procedures were employed.

Treatment with nitazoxanide 600 mg orally twice daily for five days was associated with an 85% reduction in the rate of progression to severe illness (7/195 vs. 1/184, p=0.07). All severe illnesses were clinically meaningful with six of the eight requiring hospitalization. Each of the eight severe illnesses occurred between study days 3 and 10 in subjects at high risk of severe illness according to CDC criteria. While the numbers of events observed are low, they are quantitatively and qualitatively similar or superior to those used to support emergency use of the first monoclonal antibodies for treating mild or moderate COVID-19 in the United States^{2,3,23,24}.

These findings are supported by another multicenter, randomized, double-blind, placebo-controlled study recently in subjects hospitalized with moderate to severe COVID-19 where treatment with nitazoxanide 600 mg twice daily for seven days was associated with reductions in rates of mortality and mechanical ventilation, duration of supplemental oxygen and time to hospital discharge compared to placebo²⁰.

There is presently no consensus with respect to a symptoms-based endpoint for use in trials of therapeutics in subjects with mild or moderate COVID-19. For this trial, we used the FLU-PRO Plus questionnaire to collect symptom data over 21 days and an endpoint defined based upon evidence that it is meaningful to patients. Treatment with nitazoxanide was associated with a three- to five-day reduction of the duration of illness in subjects with mild illness at baseline, but not in those with moderate illness. Given present knowledge of the stages/phases of COVID-19 disease and their relevance to treatment decisions, it is reasonable that patients with mild illness may achieve full recovery more rapidly following an effective treatment than those with moderate illness. The subgroup with mild illness at baseline was a larger and more homogenous population and likely associated with less variability in time to full recovery than subjects with moderate illness. We note that none of the nitazoxanide-treated subjects with moderate illness at baseline progressed to severe illness compared to two subjects receiving placebo.

We did not observe differences between treatment groups in qualitative or quantitative SARS-CoV-2 RNA in nasopharyngeal swabs on study day 4 or 10. While others have reported modest reductions of quantitative or qualitative RNA in nasopharyngeal swabs at different points after the end of treatment with nitazoxanide,^{20,25,26} the methods used for collection, sample handling and measuring viral loads from nasopharyngeal swabs in large multi-center clinical trials have not been validated or shown to be predictive at the patient- or trial-level of viral load, inflammation or symptoms in the lungs or clinical outcomes. It is also unclear whether RT-PCR accurately measures infectious virus, since viral RNA may persist for some time, even in the absence of replication-competent virus. This may be a particularly important limitation in the context of a host-directed therapeutic like nitazoxanide that affects assembly of the virus.

In this study, nitazoxanide was safe and well tolerated, consistent with its well-established safety profile. Safety will be an important attribute for a therapeutic for mild or moderate COVID-19.

Preventing the progression to severe COVID-19 illness is a key factor for controlling the pandemic. Only monoclonal antibodies directed to the viral spike protein have shown promise when used at the early stage of infection, yet the emergence of SARS-CoV-2 resistance to these antibodies has already been observed, necessitating the development of antibody cocktails or rescission of emergency use authorization²⁻⁴. In this study, nitazoxanide reduced the progression to severe COVID-19 and hospitalization in a manner similar to that of the monoclonal antibodies.

Persons in rural and/or economically disadvantaged communities as well as racial and ethnic minorities in the U.S. and worldwide have been disproportionately impacted by the COVID-19 pandemic for numerous reasons. An oral shelf-stable drug for the treatment of COVID-19 that can be administered at home in these vulnerable populations may mitigate the burden of reduced access to healthcare resources such as an infusion center or pharmacy with cold storage that is necessary for administration of monoclonal antibodies.

The evidence provided by this study is meaningful, reliable and consistent with expectations of a therapy that may provide potential benefit to patients at high risk of progression to severe COVID-19 and/or hospitalization. Nonetheless, these results should be confirmed with larger trials. The availability of a safe, oral, scalable, host-directed antiviral for the early treatment of COVID-19 could play an important role in reducing the number of severe illnesses and hospitalizations during this ongoing major public health crisis.

### REFERENCES

1 Kim P S, et al. 2020; 324:2149-50.
2. Bamlanivimab and Etesevimab Fact Sheet for Health Care Providers Emergency Use Authorization. Eli Lilly, Indianapolis, IN. Available at: http://pi.lilly.com/eua/bam-and-ete-eua-factsheet-hcp.pdf.
3. REGEN-COV Fact Sheet for Health Care Providers Emergency Use Authorization. Regeneron Pharmaceuticals, Inc., Tarrytown, NY. Available at: https://www.fda.gov/media/145611/download.
4. Chen R E, et al. Nat Med. 2021 April; 27(4):717-726
5. Rossignol J F. Antiviral Res 2014; 110:94-103.
6. Cao J, et al. Antiviral Res 2015; 114:1-10.
7. Rossignol J F. J Infect Public Health 2016; 9:227-30.
8. Rossignol J F, van Baalen C. [Abstract]. Presented at the 2nd International Meeting on Respiratory Pathogens. Singapore, Mar. 7-9, 2018.
9. Hong S K, et al. Int Immunopharmacol 2012; 13:23-27.
10. Wang M, et al. Cell Res 2020; 30:269-71.
11. Riccio A, et al. bioRxiv 2021 Apr. 12: 04.12.439201.
12. Bobrowski T, et al. Mol Ther 2021 Feb. 3; 29(2):873-85.
13. Mostafa A, et al. 2020 Dec. 4; 13(12):443.
14. Lian E, et al. bioRxiv 2020 Nov. 26: 11.25.399055.
15. Rossignol J F, et al. J Biol Chem 2009; 284:29798-808.
16. Piacentini S, et al. Sci Rep 2018; 8:10425.
17. Korba B E, et al. Antimicrob Agents Chemother 2008; 52:4069-71.
18. Landolt G, et al. Forum Infect Dis, 2016; 3(S1):5136.
19. Risner K H, et al. bioRxiv 2020 Aug. 13: 2020.08.12.246389.
20. Blum V F, et al. https://doi.org/10.1016/j.eclinm.2021.100981.
21. Powers J H, et al. PLoS One 2018; 13:e0194180.
22. Haffizulla J, et al. Lancet Infect Dis 2014; 14:609-18.
23. Chen P, et al. N Engl J Med 2021; 384:229-37.
24. Weinreich D M, et al. N Engl J Med 2021; 384:238-51.
25. Rocco P R M, et al. Eur Respir J. 2021 14; 2003725.
26. Silva M, et al. medRxiv 2021 Mar. 5: 03.03.21252509.

### Supplementary Material

Definition of Subjects at Risk of Severe Illness per United States Center for Disease Control and Prevention (CDC):

Subjects who are ≥65 years of age; subjects with chronic obstructive pulmonary disease (COPD), Type 2 diabetes mellitus, obesity (Body Mass Index (BMI)≥30), chronic kidney disease, sickle cell disease, serious heart conditions (such as heart failure, coronary artery disease, or cardiomyopathies), asthma (moderate or severe), cerebrovascular disease, cystic fibrosis, hypertension or high blood pressure, immunocompromised state (due to immune deficiencies, Human Immunodeficiency Virus (HIV), use of corticosteroids, or use of other immune-weakening medications), neurologic conditions (e.g., dementia), liver disease, pulmonary fibrosis, past or present history of smoking, thalassemia, or type 1 diabetes mellitus.

Although the foregoing refers to particular preferred embodiments, it will be understood that the present invention is not so limited. It will occur to those of ordinary skill in the art that various modifications may be made to the disclosed embodiments and that such modifications are intended to be within the scope of the present invention.

All of the publications, patent applications and patents cited in this specification are incorporated herein by reference in their entirety.

1. A method of treating an illness caused by a virus belonging to the Coronaviridae family, comprising administering to a subject in need thereof an effective amount of a thiazolide agent to produce an effective concentration of tizoxanide in a plasma of the subject, wherein the subject displays one or more symptoms of the illness.

2. The method of claim 1, wherein the one or more symptoms further comprise (a) one or more respiratory symptom selected from the group consisting of cough, sore throat, and nasal congestion and/or one or more constitutional symptoms selected from the group consisting of fatigue, headache, myalgia, and feverishness.

3. (canceled)

4. The method of claim 1, wherein the one or more symptoms comprises an oral temperature of no less than 37.3° C.

5. (canceled)

6. The method of claim 1, wherein the virus belonging to the Coronaviridae family is the sole cause of the illness.

7. The method of claim 1, wherein the virus belongs to the Alphacoronavirus genus or to the Betacoronavirus genus.

8. (canceled)

9. The method of claim 1, wherein the virus is selected from the group consisting of NL63 coronavirus, HKU1 coronavirus, 229E coronavirus, and OC43 coronavirus.

10. A method of preventing a viral respiratory illness, comprising administering to a subject, who does not display a symptom of the viral respiratory illness, an effective amount of a thiazolide agent to produce an effective concentration of tizoxanide in a plasma of the subject.

11. The method of claim 10, wherein the viral respiratory illness is caused by a virus belonging to the Coronaviridae family.

12. A method of treating a mild or moderate illness caused by severe acute respiratory syndrome coronavirus 2 (SARS-COV-2), comprising administering to a subject in need thereof an effective amount of a thiazolide agent to produce an effective concentration of tizoxanide in a plasma of the subject.

13. The method of claim 12, wherein said administering reduces progression of the subject from the mild or moderate illness to a severe illness and/or reduces a symptom duration of the mild or moderate illness.

14. (canceled)

15. The method of claim 12, wherein a first event of said administering is performed within 72 hours of a symptom onset of the mild or moderate illness in the subject.

16. (canceled)

17. The method of claim 1, wherein the thiazolide agent comprises nitazoxanide, tizoxanide or a pharmaceutically acceptable salt thereof.

18-20. (canceled)

21. The method of claim 17, wherein said administering comprises administering a pharmaceutical composition comprising nitazoxanide.

22. The method of claim 21, wherein the pharmaceutical composition is an extended release pharmaceutical composition.

23. The method of claim 21, wherein the pharmaceutical composition is administered orally.

24. The method of claim 21, wherein a daily dose of nitazoxanide is about 1200 mg.

25. The method of claim 24, wherein the daily dose is administered in two administering events per day.

26. The method of claim 24, wherein said administering is performed for at least 5 days.

27. The method of claim 1, wherein said administering reduces a time from a first administering event for the thiazolide compound till the subject returns to usual health.

28. The method of claim 1, wherein said administering reduces a time from a first administering event for the thiazolide compound till the subject is able to perform normal activities.
